SEQUENCE LISTING (2 sequences)

SEQ ID NO: 1 - RNA, 36 nt, synthetic construct
  source 1..36: mol_type = other RNA
gtaactagagatccctcagacccttttagtcagaat

SEQ ID NO: 2 - DNA, 25 bp, synthetic construct
  source 1..25: mol_type = other DNA
ttctgactaaaagggtctgagggat

(19) United States
(12) Patent Application Publication
(10) Pub. No.: US 2025/0186467 A1
(43) Pub. Date: Jun. 12, 2025

(54) METHODS OF TREATING MEDICAL CONDITIONS USING STAMPIDINE OR A RELATED COMPOUND

(71) Applicant: ROME Therapeutics, Inc., Boston, MA (US)

(72) Inventors: Donna L. Romero, Chesterfield, MO (US); Dennis Zaller, Lexington, MA (US); Rosana Kapeller-Libermann, Chestnut Hill, MA (US)

(21) Appl. No.: 18/846,066

(22) PCT Filed: Mar. 15, 2023
(86) PCT No.: PCT/US2023/064366

§ 371 Date: Sep. 11, 2024

Related U.S. Application Data
(60) Provisional application No. 63/319,893, filed on Mar. 15, 2022.
(60) Provisional application No. 63/424,762, filed on Nov. 11, 2022.

(51) Int. Cl. A61K 31/675; A61K 31/513; A61P 37/06

(52) U.S. Cl. CPC A61K 31/675; A61K 31/513; A61P 37/06

(57) ABSTRACT

The invention provides methods and compositions for treating medical disorders, such as cancer, an autoimmune disorders, and/or neurological disorders, and modulating LINE1 reverse transcriptase and/or HERV-K reverse transcriptase using stampidine or a related compound.

### CROSS-REFERENCE TO RELATED APPLICATIONS

This application claims the benefit of and priority to U.S. Provisional Patent Application Ser. No. 63/424,762, Nov. 11, 2022, and U.S. Provisional Patent Application Ser. No. 63/319,893, filed Mar. 15, 2022; the contents of each of which are hereby incorporated by reference in their entirety.

### REFERENCE TO AN ELECTRONIC SEQUENCE LISTING

This application contains a Sequence Listing which has been submitted electronically via Patent Center in XML format and is hereby incorporated by reference in its entirety. Said XML copy, created on Mar. 10, 2023, is named 198106_seqlist.xml and is 2,661 bytes in size.

### FIELD OF THE INVENTION

The invention provides methods and compositions for treating medical disorders, such as cancer, autoimmune disorders, and/or neurological disorders, and modulating LINE1 reverse transcriptase and/or HERV-K reverse transcriptase using stampidine or a related compound.

### BACKGROUND

Transposable elements (or transposons) are genomic DNA sequences that have the ability to move within the genome which leads to altering its organization, increase its size and creates duplications and redundancy. (Ukadike and Mustelin, J. Clin. Med., 10:856 (2021)). These genomic sequences are believed to have been introduced into the human genome by either an infection by exogenous retroviruses that infected human ancestors millions of years ago or ancient descendants of retroviruses which retained the ability to embed and replicate in human germline genome. (Ukadike and Mustelin, 2021).

Long Interspersed Nuclear Element 1 (LINE-1) are class I transposable elements in the DNA of some organisms and comprise about 17% of the human genome. LINE-1 harbors two open reading frames, ORF1 and ORF2, which in turn respectively encode ORF1p, which has nucleic acid chaperone activity, and ORF2p, with reverse transcriptase (RT) and endonuclease activities. (Reviewed in Babushok and Kazazian, Hum. Mut. 28:527-539, (2007)). LINE-1 retrotrasposition activity is mediated by ORF2p. The majority of LINE-1 elements in the human genome contain inactivating mutations, but a small percentage of LINE-1 elements are intact and have retained the ability to retrotranspose. This ability varies both among individuals and among cell types within an individual. Active LINE-1 elements are thought to disrupt the genome through insertions, deletions, rearrangements, and recombinations. (Garcia-Perez et al, Development, 143:4101-4114 (2016)). LINE-1 activity is normally tightly regulated in the germline by DNA methylation, histone modifications, and piRNA.

Retrotransposons are transposable elements which are associated with the pathogenesis of many diseases such as cancer, autoimmune disease, and neurological disorders, among others. (Zhang, et al, Frontiers in Cell and Dev. Bio., 8:657 (August 2020); Kuriyama et al, Nature: Scientific Reports, 11:23146 (2021)). LINE-1 RNA and protein overexpression can promote apoptosis, DNA damage and repair, and cellular plasticity, which can promote tumor progression. Furthermore, DNA damage caused by repetitive sequences (genome-wide or interspersed) hypomethylation can induce an inflammatory microenvironment. (Zhang, 2020).

Cancer continues to be a significant health problem despite the substantial research efforts and scientific advances reported in the literature for treating this disease. Solid tumors, including prostate cancer, breast cancer, and lung cancer remain highly prevalent among the world population. Leukemias and lymphomas also account for a significant proportion of new cancer diagnoses. Current treatment options for these cancers are not effective for all patients and/or can have substantial adverse side effects. New therapies are needed to address this unmet need in cancer therapy.

High LINE-1 activity has been found in many tumor tissues. LINE-1 mediated gene rearrangement can trigger oncogene amplification. Additionally, LINE-1 can mediate the deletion of tumor suppressor genes (Zhange, 2020). Inhibition of LINE-1 RT in cancer cells, either via RNA interface-dependent silencing of active LINE-1 elements or using RT inhibitory compounds, can reduce cancer cell proliferation, promote cancer cell differentiation and can retard tumor progression in certain animal models. (Sciamann et al, Frontiers in Chemistry, 4:6 (February 2016)).

LINE-1 RT uses a procedure termed target-site-primed reverse transcription (TPRT) which involves nicking of the genomic DNA, followed by reverse transcription and insertion of LINE-1 into the genome. The products of LINE-1 reverse transcription are potential triggers of DNA sensing receptors such as cGAS, which is a DNA sensor that activates the STING pathway, leading to type I interferon production. (Zhao, J. Autoimmunity, 90:105-115 (2018)). Hypomethylated and highly expressed LINE-1 has been found in many patients with autoimmune diseases such as systemic lupus erythematosus (SLE), Sjögren's syndrome (SS), and psoriasis. (Zhang et al). LINE-1 has also been found to be significantly upregulated in patients with dermatomyositis (DM), which patients also showed significantly elevated levels of interferon α and interferon β. (Kuriyama et al, J. Am. Acad, Dermatol., 84(4):1103-1105 (2020)).

Interferon overproduction is a characteristic feature of type I interferonopathies. These include rare genetic diseases with occurrence rates from 1:10,000 to 1:1,000,000. Pathological overexpression of type I interferon causes immune system hyperactivation that leads to systemic inflammation which can affect the nervous system, lung, and blood vessels, among other organ systems. (Nesterova et al. “Congenital and Acquired Interferonopathies: Differentiated Approaches to Interferon Therapy”. Innate Immunity in Health and Disease, Ed. Saxena and Prakash, IntechOpen, 2020). LINE-1 expression has been shown to induce type I interferons, which lead to type I interferonopathies. (Ukadike and Mustelin, 2021). These diseases have very limited effective treatment options, so there is a high unmet medical need in this area.

LINE-1 expression is high in brain tissue as compared to other organs. LINE-1 is active in neural progenitor cells, and overexpression of LINE-1 increases somatic mosaicism. LINE-1 has also been implicated in neurological disorders such as ataxia telangiectasia (AT) and Rett syndrome. LINE-1 is also implicated in the aging process and frontotemporal lobe degeneration. (Zhang, 2020).

Human endogenous retroviruses (HERVs) comprise nearly 8% of the human genome and are believed to be derived from ancient integrations of retroviruses into the germline. The biology of HERVs is poorly defined, but there is accumulating evidence supporting pathological roles in diverse diseases such as cancer, autoimmune, and neurodegenerative diseases. Functional proteins are produced by HERV-encoded genes including reverse transcriptases (RTs), which could be a contributor to the pathology attributed to aberrant HERV-K expression.

HERVs play a role in early development by rewiring the gene regulatory network of the preimplantation embryo (Fu et al, Biomolecules, 11(6):829 (2021)). HERV expression appears to be a hallmark of the undifferentiated state, the acquisition of phenotypic plasticity and stem cell character (Balestrieri et al, Frontiers in Microbiology, 9:1448 (2018)); traits associated with aggressive cancer and poor patient outcomes. HERV expression is normally tightly controlled in normal adult tissues but is reported to be aberrantly expressed in cancer (Downey et al, Int. J. Cancer, 137(6):1249-1257 (2015)), inflammatory diseases (Greenig, PeerJ 7:e6711 (2019)), neurological diseases (Kury et al, Trends Mol. Med., 24(4):379-394 (2018)), aging (Gorbunova et al, Nature, 596(7870):43-53 (2021)), and viral disease (Romer, Frontiers in Neuroscience, 15:648629-648629 (2021)). There are numerous reports of upregulation of HERV-K [HML-2 (human endogenous MMTV-like) subtype] derived mRNA and protein in a variety of solid and liquid tumor types (Dervan et al, Front. Onc., 11:658489 (2021); Hohn et al, Front. Onc., 3:246 (2013)). The disease association with endogenous retroviruses and the expression of HERV encoded proteins during disease states suggests that anti-retroviral therapy could be explored in the management of these conditions.

Accordingly, the need exists for new therapeutic methods that provide improved efficacy and/or reduced side effects for treating medical disorders, such as cancer, autoimmune disease, and/or neurological disorders. The present invention addresses the foregoing needs and provides other related advantages.

### SUMMARY

The invention provides methods and compositions for treating medical disorders, such as cancer, autoimmune disorders, and/or neurological disorders, and modulating LINE1 reverse transcriptase and/or HERV-K reverse transcriptase using stampidine or a related compound. In particular, one aspect of the invention provides a method of treating a disorder selected from the group consisting of an autoimmune disorder and a neurological disorder. The method comprises administering to a subject in need thereof a therapeutically effective amount of a compound of Formula I to treat the disorder; wherein Formula I is represented by:

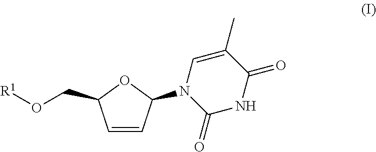

or a pharmaceutically acceptable salt thereof; where the variables are as defined in the detailed description. Further description of additional collections of stampidine and related compounds useful in the method are described in the detailed description. The compounds may be part of a pharmaceutical composition comprising a pharmaceutically acceptable carrier. Additional features of the method are described in the detailed description.

Another aspect of the invention provides a method of treating a disorder that is cancer. The method comprises administering to a subject in need thereof a therapeutically effective amount of a compound of Formula I to treat the disorder. Additional features of the method are described in the detailed description.

Another aspect of the invention provides a method of inhibiting LINE1 reverse transcriptase activity in a subject suffering from a disorder selected from the group consisting of an autoimmune disorder and a neurological disorder. The method comprises contacting a LINE1 reverse transcriptase with an effective amount of a compound of Formula I, in order to inhibit the activity of said LINE1 reverse transcriptase; wherein Formula I is represented by:

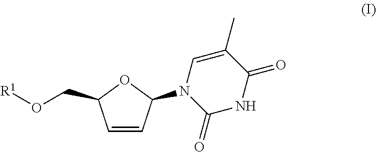

or a pharmaceutically acceptable salt thereof; where the variables are as defined in the detailed description. Further description of additional collections of stampidine and related compounds useful in the method are described in the detailed description. Additional features of the method are described in the detailed description.

Another aspect of the invention provides a method of inhibiting LINE1 reverse transcriptase activity in a subject suffering from a disorder that is cancer. The method comprises contacting a LINE1 reverse transcriptase with an effective amount of a compound of Formula I, in order to inhibit the activity of said LINE1 reverse transcriptase. Additional features of the method are described in the detailed description.

Another aspect of the invention provides a method of inhibiting HERV-K reverse transcriptase activity in a subject suffering from a disorder selected from the group consisting of an autoimmune disorder and a neurological disorder. The method comprises contacting a HERV-K reverse transcriptase with an effective amount of a compound of Formula I, in order to inhibit the activity of said HERV-K reverse transcriptase; wherein Formula I is represented by:

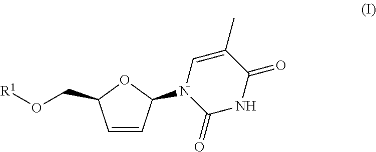

or a pharmaceutically acceptable salt thereof; where the variables are as defined in the detailed description. Further description of additional collections of stampidine and related compounds useful in the method are described in the detailed description. Additional features of the method are described in the detailed description.

Another aspect of the invention provides a method of inhibiting HERV-K reverse transcriptase activity in a subject suffering from a disorder that is cancer. The method comprises contacting a HERV-K reverse transcriptase with an effective amount of a compound of Formula I, in order to inhibit the activity of said HERV-K reverse transcriptase. Additional features of the method are described in the detailed description.

### BRIEF DESCRIPTION OF FIGURES

FIG. 1 is a graph depicting inhibition of LINE1 reverse transcriptase by a test compound in an artificial-intron Cis LINE1 reporter assay, as described in Example 1.

FIGS. 2A and 2B show, in the in vivo decitabine challenge model described in Example 5, that repeated dosing of decitabine induces interferon-stimulated gene (ISG) response in the spleen in the control animals.

FIG. 3 depicts the effects of repeated administration of stampidine in the in vivo decitabine challenge model, as described in Example 5.

FIGS. 4A and 4B show two exemplary PBMC donors' responses to decitabine-induced interferon levels upon administration of stampidine, as described in Example 6. FIG. 4A depicts a representative high responder, and FIG. 4B depicts a representative moderate responder.

FIG. 5 depicts levels of pTBK1 observed in HaCaT cells treated with stampidine (labeled “Compound 1”) or a DMSO control, with or without exposure to UVB light, as described in Example 7.

FIGS. 6A and 6B are graphs depicting inhibition of IFN by stampidine (FIG. 6A) and stavudine (FIG. 6B) in the cellular assay for altering IFN production in THP1 TREX1 KO cells, as described in Example 8.

### DETAILED DESCRIPTION

The invention provides methods and compositions for treating medical disorders, such as cancer, autoimmune disorders, and/or neurological disorders, and modulating LINE1 reverse transcriptase and/or HERV-K reverse transcriptase using stampidine or a related compound.

Various aspects of the invention are set forth below in sections; however, aspects of the invention described in one particular section are not to be limited to any particular section. Further, when a variable is not accompanied by a definition, the previous definition of the variable controls.

### Definitions

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C_{1_4}alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. For example, certain compounds herein may have one, two or three chiral centres and the invention encompasses pure chiral forms and mixtures thereof in any proportion. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. According to a further aspect of the invention there is provided a compound of the Formula (I), or a pharmaceutically-acceptable salt thereof, substantially free of other stereoisomers, such as wherein said compound of Formula I, or pharmaceutically acceptable salt thereof, has an enantiomeric excess (% ee) of ≥90%, ≥95%, ≥98% or ≥99%. In one embodiment, the compound of formula (I), or a pharmaceutically acceptable salt thereof, is present in a form having an enantiomeric excess (% ee) of ≥95%.

Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

The terms “a” and “an” as used herein mean “one or more” and include the plural unless the context is inappropriate.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof. For example, treatment of cancer may mean prolonging the period of time where tumor burden does not increase (progression free survival), reduction of the tumor burden, extension of the overall survival time of a patient, amelioration of symptoms associated with the cancer, prevention of metastasis, slowing of metastasis, and the like. Treatment of an autoimmune disease includes reduction of the symptoms of the disease, extension of time between disease flare-ups, remission of disease, prevention of worsening of the disease, and the like. Treatment of neurological disease may include improvement of cognitive function, reduction of the rate of cognitive loss, reduction of symptoms, and the like.

In some embodiments, treatment is administered after one or more symptoms have developed. In some embodiments, treatment is administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the term “pharmaceutical composition” refers to the combination of an active agent with a carrier, adjuvant, or vehicle, inert or active, making the composition especially suitable for diagnostic or therapeutic use in vivo or ex vivo.

As used herein, the term “pharmaceutically acceptable carrier, adjuvant, and/or vehicle” refers to any non-toxic carrier, adjuvant and/or vehicle that does not destroy the pharmacological activity of the compound with which it is formulated. Pharmaceutically acceptable carriers, adjuvants and/or vehicles that are used in the compositions of this invention include, but are not limited to, ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as potassium sorbate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, emulsions (e.g., such as an oil/water or water/oil emulsions), and various types of wetting agents. The compositions also can include stabilizers and/or preservatives. For examples of carriers, vehicles and adjuvants, see e.g., Martin, Remington's Pharmaceutical Sciences, 15th Ed., Mack Publ. Co., Easton, PA [1975].

The term “combination” refers to simultaneous, separate, or sequential administration. In one aspect of the invention, “combination” refers to simultaneous administration. In another aspect of the invention, “combination” refers to separate administration. In another aspect of the invention, “combination” refers to sequential administration. Where the administration is separate or sequential, the delay in administering the one or more additional therapeutic agents is done at an interval designed such as to not lose the beneficial effect of the combination.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As used herein, the term “comprising” or “comprises” is used in reference to compounds, uses, compositions, methods, and respective component(s) thereof that are essential to the method or composition, yet open to the inclusion of unspecified elements, whether essential or not. The term “consisting of” refers to compounds, uses, compositions, methods, and respective component(s) thereof as described herein which are exclusive of any element not recited in that description of the embodiment. The term “consisting essentially of” refers to those elements required for a given embodiment. The term permits the presence of elements that do not materially affect the basic and novel functional characteristic(s) of that embodiment.

As used herein, “stampidine” refers to methyl ((S)-(4-bromophenoxy)(((2S,5R)-5-(5-methyl-2,4-dioxo-3,4-dihydropyrimidin-1(2H)-yl)-2,5-dihydrofuran-2-yl)methoxy)phosphoryl)-L-alaninate, which is the chemical entity represented by the structure:

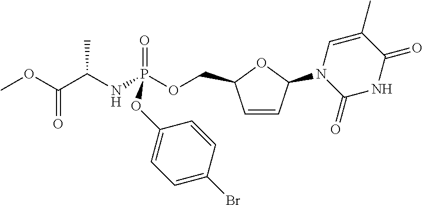 [SMILES: COC(=O)[C@H](C)N[P@](=O)(OC[C@@H]1C=C[C@H](n2cc(C)c(=O)[nH]c2=O)O1)Oc1ccc(Br)cc1]

or a pharmaceutically acceptable salt thereof, in any physical form, e.g., crystalline or amorphous. Alternative names for stampidine include STAMP, DDE-113, and HI-113. The term “stampidine” includes any commercially relevant formulations which include methyl ((S)-(4-bromophenoxy)(((2S,5R)-5-(5-methyl-2,4-dioxo-3,4-dihydropyrimidin-1(2H)-yl)-2,5-dihydrofuran-2-yl)methoxy)phosphoryl)-L-alaninate, or a pharmaceutically acceptable salt thereof. The synthesis of stampidine starting, for example, from commercially available 2′,3′-didehydro-3′-deoxythymidine, are provided in, for example, U.S. Pat. No. 6,350,736.

As used herein, “stavudine” refers to 1-((2R,5S)-5-(hydroxymethyl)-2,5-dihydrofuran-2-yl)-5-methylpyrimidine-2,4(1H,3H)-dione, which is the chemical entity represented by the structure:

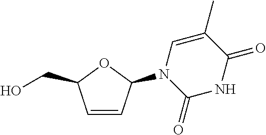 [SMILES: Cc1cn([C@H]2C=C[C@@H](CO)O2)c(=O)[nH]c1=O]

or a pharmaceutically acceptable salt thereof, in any physical form, e.g., crystalline or amorphous. Alternative names for stavudine include Zerit, STV, d4T, 2′,3′-didehydro-3′-deoxythymidine, and BMY-27857. The term “stavudine” includes any commercially relevant formulations which include 1-((2R,5S)-5-(hydroxymethyl)-2,5-dihydrofuran-2-yl)-5-methylpyrimidine-2,4(1H,3H)-dione, or a pharmaceutically acceptable salt thereof. Stavudine is a commercially available compound.

As a general matter, compositions specifying a percentage are by weight unless otherwise specified.

### I. Methods of Treating Medical Disorders

It is contemplated that the stampidine and related compounds described herein, such as a compound of Formula I or other compounds in Section III, below, provide therapeutic benefits to subjects suffering from cancer, autoimmune disease, and/or neurological disorders.

### Methods of Treating an Autoimmune Disorder or a Neurological Disorder

Accordingly, one aspect of the invention provides a method of treating a disorder selected from the group consisting of an autoimmune disorder and a neurological disorder. The method comprises administering to a subject in need thereof a therapeutically effective amount of a compound of Formula I to treat the disorder; wherein Formula I is represented by:

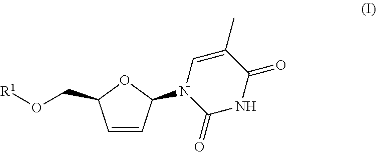

or a pharmaceutically acceptable salt thereof; wherein:
R¹ is

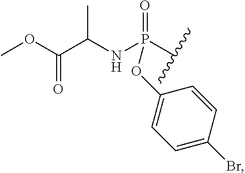 [SMILES: *P(=O)(NC(C)C(=O)OC)Oc1ccc(Br)cc1]

hydrogen, —P(O)(OH)₂, —P(O)(OH)—O—P(O)(OH)₂, or —(P(O)(OH))₂—O—P(O)(OH)₂.

In certain embodiments, the particular compound of Formula I is a compound defined by one of the embodiments described in Section III, below, such as

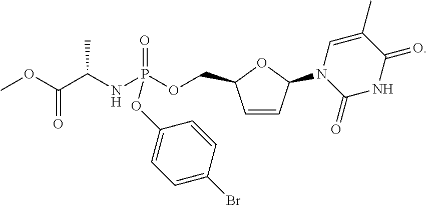 [SMILES: COC(=O)[C@H](C)NP(=O)(OC[C@@H]1C=C[C@H](n2cc(C)c(=O)[nH]c2=O)O1)Oc1ccc(Br)cc1]

In certain embodiments, the compound of Formula I, or other compound defined by one of the embodiments described in Section III, below, such as

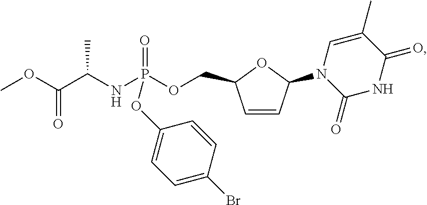 [SMILES: COC(=O)[C@H](C)NP(=O)(OC[C@@H]1C=C[C@H](n2cc(C)c(=O)[nH]c2=O)O1)Oc1ccc(Br)cc1]

is administered in a pharmaceutical composition comprising the compound and a pharmaceutically acceptable carrier, adjuvant, and/or vehicle, as further described in Section V, below.

In certain embodiments, the method further comprises administering an effective amount of an additional therapeutic agent, as further described in Section IV, below.

### Autoimmune Disorders

In certain embodiments, the disorder is an autoimmune disorder. As used herein, “autoimmune disorders” includes those disorders which are traditionally classified as autoimmune disorders, as well as inflammatory disorders and immune disorders (excluding viral infections). The intention with the term “autoimmune disorder” is to include all diseases and disorders which are driven by innate immune responses and adaptive immune responses which initiate some sort of innate immune inflammatory response. It is the applicant's intent to have the term “autoimmune disorder” include the full scope of diseases and disorders which are driven by innate inflammation, with the exception of viral infections.

In certain embodiments, the invention provides for treatment of an autoimmune disorder. Traditional autoimmune disorders commonly occur when the immune system attacks normal cells and/or tissues in the body. Inflammatory disorders often present with chronic inflammation (among other symptoms) in the absence of infection. Autoimmune disorders also include symptoms which arise when the cellular immune system reacts against the body's autoantigens. There may also be autoimmune and/or inflammation manifestation associated with a range of primary immunodeficiency diseases. In further aspects of these embodiments, the autoimmune disease or disorder is associated with high levels of LINE-1 and/or HERV-K RNA protein expression.

One embodiment of the invention is a method of treating type I interferonopathies. In one aspect of this embodiment, the type I interferonopathy is a congenital disorder associated with type I interferon overexpression. In one aspect of this embodiment, the congenital type I interferonopathy is selected from Aicardi-Goutieres syndrome (AGS), Singleton-Merten syndrome, proteasome-associated autoinflammatory syndromes, chronic atypical neutrophilic dermatosis with lipodystrophy and elevated temperature (CANDLE), STING-associated vasculopathy with onset in infancy (SAVI), Japanese autoinflammatory syndrome with lipodystrophy (JASL), spondyloenchondrodysplasia (SPENCD), ISG15 deficiency, Ubiquitin-Specific Peptidase 18 deficiency (pseudo-TORCH syndrome), chronic atypical neurophilic dermatitis with lipodystrophy, DNA II deficiency, trichoheptoenteric syndrome 2, retinal vasculopathy with cerebral leukodystrophy, familial chilblain lupus, and X-linked reticulate pigmentary disorder (XLPDR). In another embodiment, the type I interferonopathy is an acquired disorder in the IFN system.

In one embodiment, the invention provides a method of treating an autoimmune disease which results in an overproduction of interferon. In one aspect of this embodiment, the interferon expressed includes type I interferon. In another aspect of this embodiment, the autoimmune disease is associated with elevated LINE-1 activity and/or expression. In another aspect of this embodiment, the autoimmune disease is associated with elevated HERV-K RNA activity and/or expression.

In certain embodiments, the autoimmune disorder is selected from the group consisting of achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Balo disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifactorial osteomyelitis (CRMO), Churg-Strauss syndrome or eosinophilic granulomatosis, cicatricial pemphigoid, Cogan's syndrome, cold agglutinin disease, complex regional pain syndrome (previously called reflex sympathetic dystrophy), congenital heart block, coxsackle myocarditis, CREST syndrome, Crohn's disease, cutaneous lupus erythematosus (CLE), dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, graft versus host disease, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (acne inversa), inflammatory bowel disease, IgA nephropathy, IgG4-related sclerosing disease, immune thrombocytopenic pupura (ITP), inclusion body myositis (IBM), interstitial cystitis (IC), juvenile arthritis, juvenile diabetes (type I diabetes), juvenile myositis (JM), Kawasaki disease, Lambert-Eaton syndrome, lichen planus, lichen sclerosus, ligneous conjunctivitis, linear IgA disease (LAD), lupus nephritis, lyme disease (chronic), Meniere's disease, microscopic polyangiitis (MPA), mixed connective tissue disease (MCTD), multifocal motor neuropathy, multiple sclerosis, myasthenia gravis, myelin oligodendrocyte glycoprotein antibody disorder, myositis, narcolepsy, neonatal lupus, neutropenia, ocular cicatricial pemphigoid, optic neuritis, palindromic rheumatism, pediatric autoimmune neuropsychiatric disorders associated with streptococcus infections (PANDAS), paraneoplastic cerebellar degeneration (PCD), paroxysmal nocturnal hemoglobinuria (PNH), pars planitis (peripheral uveitis), Parsonage-Turner syndrome, pemphigus, peripheral neuropathy, perivenous encephalomyelitis, pernicious anemia (PA), POEMS syndrome, polyarteritis rheumatica, polymyositis, postmyocardial infarction syndrome, postpericardiotomy syndrome, primary biliary cholangitis, primary sclerosing cholangitis, progesterone dermatitis, progressive hemifacial atrophy (Parry Romberg syndrome), psoriasis, psoriatic arthritis, pure red cell aplasia, pyoderma gangrenosum, Raynoud's phenomena, reactive arthritis, relapsing polychondritis, restless leg syndrome (RLS), retroperitoneal fibrosis, rheumatic fever, rheumatoid arthritis (RA), sarcoidosis, Schmidt syndrome (autoimmune polyendocrine syndrome type II), scleritis, scleroderma, Sjögren's disease, stiff person syndrome, Susac's syndrome, sympathetic ophthalmia, systemic lupus erythematosus (SLE), Takayasu's arteritis, thrombotic thrombocytopenic pupura, thyroid eye disease, Tolosa-Hunt syndrome, transverse myelitis, ulcerative colitis, undifferentiated connective tissue disease, uveitis, vasculitis, vitiligo, Vogt-Koyanagi-Harada disease, and warm autoimmune hemolytic anemia.

In certain embodiments, the autoimmune disorder is selected from Aicardi-Goutieres syndrome, rheumatoid arthritis, psoriasis, systemic lupus erythematosus (SLE), cutaneous lupus erythematosus (CLE), graft versus host disease, scleroderma, type I diabetes, dermatomyositis, inflammatory bowel disease, ulcerative colitis, Crohn's disease, vasculitis, and Sjögren's syndrome.

In certain embodiments, the autoimmune disorder Aicardi-Goutieres syndrome (AGS). In another embodiment, the autoimmune disorder is systemic lupus erythematosus (SLE). In another embodiment, the autoimmune disease is lupus nephritis. In a further embodiment, the autoimmune disease is cutaneous lupus erythematosus (CLE). In another embodiment, the autoimmune disease is dermatomyositis.

In certain embodiments, the autoimmune disorder is a type 1 interferonopathy. In certain embodiments, the autoimmune disorder is type 1 diabetes, Aicardi-Goutieres syndrome (AGS), systemic lupus erythematosus (SLE), lupus nephritis, cutaneous lupus erythematosus (CLE), familial chilblain lupus, systemic sclerosis, STING-associated vasculopathy with onset in infancy (SAVI), Sjögren's syndrome, or dermatomyositis. In certain embodiments, the immune disorder is a type 1 interferonopathy, type 1 diabetes, Aicardi-Goutieres syndrome (AGS), systemic lupus erythematosus (SLE), lupus nephritis, cutaneous lupus erythematosus (CLE), dermatomyositis, or Sjogren's syndrome. In certain embodiments, the autoimmune disorder is systemic lupus erythematosus (SLE), lupus nephritis, cutaneous lupus erythematosus (CLE), or familial chilblain lupus.

In certain embodiments, the autoimmune disorder is type 1 diabetes. In certain embodiments, the autoimmune disorder is familial chilblain lupus. In certain embodiments, the autoimmune disorder is systemic sclerosis. In certain embodiments, the autoimmune disorder is STING-associated vasculopathy with onset in infancy (SAVI). In certain embodiments, the autoimmune disorder is Sjögren's syndrome.

In certain embodiments, the autoimmune disorder is inflammatory bowel disease, Crohn's disease, or ulcerative colitis. In certain embodiments, the autoimmune disorder is inflammatory bowel disease. In certain embodiments, the autoimmune disorder is Crohn's disease. In certain embodiments, the autoimmune disorder is ulcerative colitis. In one embodiment, the autoimmune disorder is drug-induced colitis, such as colitis associated with the administration of checkpoint inhibitors to cancer patients.

In certain embodiments, the autoimmune disorder is osteoarthritis, nonalcoholic steatohepatitis (NASH), non-alcoholic fatty liver disease (NAFLD), cholestatic liver disease, sclerosing cholangitis, asthma, bronchitis, chronic obstructive pulmonary disease (COPD), pulmonary fibrosis, pulmonary hypertension, pericarditis, gout, or myositis.

In certain embodiments, the autoimmune disorder is Reiter's syndrome, exfoliative psoriatic dermatitis, pemphigus vulgaris, autoimmune uveitis, pulmonary hemosiderosis, amyloidosis, aphthous stomatitis, thyroiditis, gastritis, adrenalitis (Addison's disease), ovaritis, primary biliary cirrhosis, myasthenia gravis, gonadal failure, hypoparathyroidism, alopecia, malabsorption syndrome, pernicious anemia, hepatitis, hypopituitarism, diabetes insipidus, or sicca syndrome.

### Neurological Disorders

In certain embodiments, the disorder is a neurological disorder. In certain embodiments, the neurological disorder is Alzheimer's disease, amyotrophic lateral sclerosis (ALS), multiple sclerosis, Parkinson's disease, Huntington's disease, peripheral neuropathy, age-related macular degeneration, Creutzfeldt-Jacob disease, stroke, prion disease, frontotemporal dementia, Pick's disease, progressive supranuclear palsy, spinocerebellar ataxias, Lewy body disease, dementia, multiple system atrophy, epilepsy, bipolar disorder, schizophrenia, an anxiety disorder, or major depression. In certain embodiments, the neurological disorder is Alzheimer's disease, amyotrophic lateral sclerosis (ALS), multiple sclerosis, Parkinson's disease, Huntington's disease, or dementia. In another embodiment, the neurological disorder is ALS or progressive supranuclear palsy.

In certain embodiments, the neurological disorder is peripheral neuropathy, age-related macular degeneration, Creutzfeldt-Jacob disease, stroke, prion disease, frontotemporal dementia, Pick's disease, progressive supranuclear palsy, spinocerebellar ataxias, Lewy body disease, dementia, multiple system atrophy, epilepsy, bipolar disorder, schizophrenia, an anxiety disorder, or major depression.

In certain embodiments, the neurological disorder is Alzheimer's disease. In other embodiments, the neurological disorder is amyotrophic lateral sclerosis (ALS). In another embodiment, the neurological disorder is multiple sclerosis. In a further embodiment, the neurological disorder is Parkinson's disease. In another embodiment, the neurological disorder is Huntington's disease. In another embodiment, the neurological disorder is dementia. In certain embodiments, the neurological disorder is age-related macular degeneration. In a further embodiment, the neurological disorder is progressive supranuclear palsy. In certain embodiments, the neurological disorder is stroke.

### Methods of Treating Cancer

Another aspect of the invention provides a method of treating a disorder that is cancer. The method comprises administering to a subject in need thereof a therapeutically effective amount of a compound of Formula I to treat the disorder; wherein Formula I is represented by:

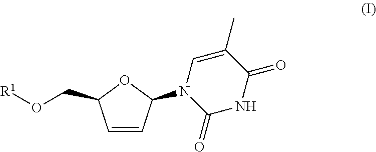

or a pharmaceutically acceptable salt thereof; wherein:
R¹ is

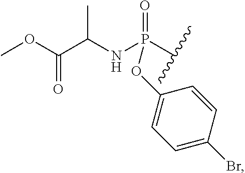 [SMILES: *P(=O)(NC(C)C(=O)OC)Oc1ccc(Br)cc1]

hydrogen, —P(O)(OH)₂, —P(O)(OH)—O—P(O)(OH)₂, or —(P(O)(OH))₂—O—P(O)(OH)₂.

In certain embodiments, the particular compound of Formula I is a compound defined by one of the embodiments described in Section III, below, such as

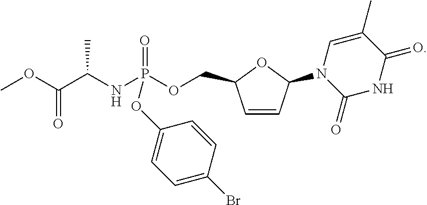 [SMILES: COC(=O)[C@H](C)NP(=O)(OC[C@@H]1C=C[C@H](n2cc(C)c(=O)[nH]c2=O)O1)Oc1ccc(Br)cc1]

In certain embodiments, the compound of Formula I, or other compound defined by one of the embodiments described in Section III, below, such as

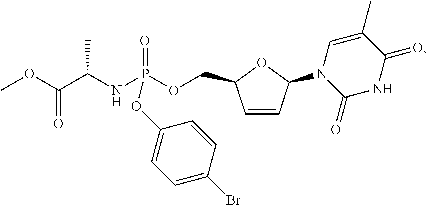 [SMILES: COC(=O)[C@H](C)NP(=O)(OC[C@@H]1C=C[C@H](n2cc(C)c(=O)[nH]c2=O)O1)Oc1ccc(Br)cc1]

is administered in a pharmaceutical composition comprising the compound and a pharmaceutically acceptable carrier, adjuvant, and/or vehicle, as further described in Section V, below.

In certain embodiments, the method further comprises administering an effective amount of an additional therapeutic agent, as further described in Section IV, below.

### Cancer

In certain embodiments, the disorder is cancer. In certain embodiments, the cancer is a solid tumor or leukemia. In certain embodiments, the cancer is a solid tumor. In certain embodiments, the cancer is a carcinoma or melanoma. In certain embodiments, the cancer is a carcinoma. In certain embodiments, the cancer is a sarcoma. In certain embodiments, the cancer is a melanoma. In certain embodiments, the cancer is a lymphoma. In certain embodiments, the cancer is a leukemia.

In certain embodiments, the cancer is breast cancer, ovarian cancer, uterine cancer, cervical cancer, prostate cancer, testicular cancer, lung cancer, leukemia, head and neck cancer, oral cancer, esophageal cancer, stomach cancer, bile duct and gallbladder cancers, bladder cancer, urinary tract cancer, colon cancer, rectal cancer, thyroid cancer, pancreatic cancer, kidney cancer, liver cancer, brain cancer, skin cancer, or eye cancer.

In certain embodiments, the cancer is breast cancer, ovarian cancer, uterine cancer, cervical cancer, prostate cancer, testicular cancer, lung cancer, leukemia, head and neck cancer, oral cancer, esophageal cancer, stomach cancer, bile duct and gallbladder cancers, bladder cancer.

In certain embodiments, the cancer has (i) expression of LINE1 RNA, LINE1 ORF1 polypeptide, and/or LINE1 ORF2 polypeptide; (ii) activity of LINE1 reverse transcriptase; (iii) expression of HERV-K RNA, and/or (iv) activity of HERV-K reverse transcriptase.

In certain embodiments, the cancer has (i) expression of LINE1 RNA, LINE1 ORF1 polypeptide, and/or LINE1 ORF2 polypeptide; and/or (ii) activity of LINE1 reverse transcriptase. In certain embodiments, the cancer has expression of LINE1 RNA, LINE1 ORF1 polypeptide, and/or LINE1 ORF2 polypeptide. In certain embodiments, the cancer has expression of LINE1 RNA. In certain embodiments, the cancer has expression of LINE1 ORF1 polypeptide. In certain embodiments, the cancer has expression of LINE1 ORF2 polypeptide. In certain embodiments, the cancer has activity of LINE1 reverse transcriptase.

In certain embodiments, the cancer has (i) expression of HERV-K RNA, and/or (ii) activity of HERV-K reverse transcriptase. In certain embodiments, the cancer has expression of HERV-K RNA. In certain embodiments, the cancer has activity of HERV-K reverse transcriptase.

In certain embodiments, the cancer has elevated (i) levels of LINE1 RNA, LINE1 ORF1 polypeptide, and/or LINE1 ORF2 polypeptide; (ii) activity of LINE1 reverse transcriptase; (iii) levels of HERV-K RNA, and/or (iv) activity of HERV-K reverse transcriptase.

In certain embodiments, the cancer has elevated (i) levels of LINE1 RNA, LINE1 ORF1 polypeptide, and/or LINE1 ORF2 polypeptide; and/or (ii) activity of LINE1 reverse transcriptase. In certain embodiments, the cancer has elevated levels of LINE1 RNA, LINE1 ORF1 polypeptide, and/or LINE1 ORF2 polypeptide. In certain embodiments, the cancer has elevated levels of LINE1 RNA. In certain embodiments, the cancer has elevated levels of LINE1 ORF1 polypeptide. In certain embodiments, the cancer has elevated levels of LINE1 ORF2 polypeptide. In certain embodiments, the cancer has elevated activity of LINE1 reverse transcriptase.

In certain embodiments, the cancer has elevated (i) levels of HERV-K RNA, and/or (ii) activity of HERV-K reverse transcriptase. In certain embodiments, the cancer has elevated levels of HERV-K RNA. In certain embodiments, the cancer has elevated activity of HERV-K reverse transcriptase.

In certain embodiments, the cancer is an epithelial cancer. In certain embodiments, the epithelial cancer is pancreatic cancer, colorectal cancer, breast cancer, prostate cancer, esophageal cancer, head and neck cancer, renal cancer, ovarian cancer, or lung cancer. In certain embodiments, the cancer is pancreatic cancer, colorectal cancer, breast cancer, prostate cancer, renal cancer, ovarian cancer, or lung cancer. In certain embodiments, the cancer is pancreatic cancer. In certain embodiments, the cancer is pancreatic adenocarcinoma. In certain embodiments, the cancer is colorectal cancer. In certain embodiments, the cancer comprises microsatellite instable (MSI) colorectal cancer or microsatellite stable (MSS) colorectal cancer. In certain embodiments, the cancer is breast cancer. In certain embodiments, the cancer is prostate cancer. In certain embodiments, the cancer is esophageal cancer. In certain embodiments, the cancer is head and neck cancer. In certain embodiments, the cancer is renal cancer. In certain embodiments, the cancer is ovarian cancer. In certain embodiments, the cancer is lung cancer. In certain embodiments, the lung cancer is non-small cell lung carcinoma or small cell lung carcinoma. In certain embodiments, the cancer is non-small cell lung carcinoma. In certain embodiments, the cancer is small cell lung carcinoma.

In certain embodiments, the cancer is a preneoplastic or early cancer lesion. In certain embodiments, the cancer is intraductal papillary mucinous neoplasm (IPMN), pancreatic intraepithelial neoplasia (PanIN), ductal carcinoma in situ (DCIS), or Barrett's Esophagus. In certain embodiments, the cancer intraductal papillary mucinous neoplasm (IPMN). In certain embodiments, the cancer is pancreatic intraepithelial neoplasia (PanIN). In certain embodiments, the cancer is ductal carcinoma in situ (DCIS). In certain embodiments, the cancer is Barrett's Esophagus.

In certain embodiments, the cancer has elevated levels of pericentrometric human satellite II (HSATII) RNA. In some embodiments, the cancer is a microsatellite instable (MSI) cancer. In some embodiments, the cancer is a microsatellite stable (MSS) cancer.

In aspects of any of the embodiments, the cancer is associated with long interspersed nuclear element-1 (LINE-1) reverse transcriptase (RT). In further aspects of these embodiments, the cancer is associated with high levels of LINE-1 RT activity.

In certain embodiments, the cancer is selected from B cell lymphomas (e.g., B cell chronic lymphocytic leukemia, B cell non-Hodgkin lymphoma, cutaneous B cell lymphoma, diffuse large B cell lymphoma), basal cell carcinoma, bladder cancer, blastoma, brain metastasis, breast cancer, Burkitt lymphoma, carcinoma (e.g., adenocarcinoma (e.g., of the gastroesophageal junction)), cervical cancer, colon cancer, colorectal cancer (colon cancer and rectal cancer), endometrial carcinoma, esophageal cancer, Ewing sarcoma, follicular lymphoma, gastric cancer, gastroesophageal junction carcinoma, gastrointestinal cancer, glioblastoma (e.g., glioblastoma multiforme, e.g., newly diagnosed or recurrent), glioma, head and neck cancer (e.g., head and neck squamous cell carcinoma), hepatic metastasis, Hodgkin's and non-Hodgkin's lymphoma, kidney cancer (e.g., renal cell carcinoma and Wilms' tumors), laryngeal cancer, leukemia (e.g., chronic myelocytic leukemia, hairy cell leukemia), liver cancer (e.g., hepatic carcinoma and hepatoma), lung cancer (e.g., non-small cell lung cancer and small-cell lung cancer), lymphoblastic lymphoma, lymphoma, mantle cell lymphoma, metastatic brain tumor, metastatic cancer, myeloma (e.g., multiple myeloma), neuroblastoma, ocular melanoma, oropharyngeal cancer, osteosarcoma, ovarian cancer, pancreatic cancer (e.g., pancreatic ductal adenocarcinoma), prostate cancer (e.g., hormone refractory (e.g., castration resistant), metastatic, metastatic hormone refractory (e.g., castration resistant, androgen independent)), renal cell carcinoma (e.g., metastatic), salivary gland carcinoma, sarcoma (e.g., rhabdomyosarcoma), skin cancer (e.g., melanoma (e.g., metastatic melanoma)), soft tissue sarcoma, solid tumor, squamous cell carcinoma, synovia sarcoma, testicular cancer, thyroid cancer, transitional cell cancer (urothelial cell cancer), uveal melanoma (e.g., metastatic), verrucous carcinoma, vulval cancer, and Waldenstrom macroglobulinemia.

In some embodiments, the cancer is a virus-associated cancer. As used herein, the term “virus-associated cancer” means any cancer in which a virus is known to play a role. For example, Epstein-Barr virus (EBV) has been reported to be associated with the endemic variant of Burkitt lymphoma and certain other lymphomas. Infection by human papilloma virus (HPV) is believed to be responsible for certain types of cervical and/or genital cancer. Human T-cell leukemia virus 1 (HTLV-1) has been reported to be linked adult T-cell leukemia/lymphoma (ATLL). Human T-cell leukemia virus 2 (HTLV-2) has been reported to be linked to cutaneous T-cell lymphoma. Human herpes virus 8 (HHV-8) is believed to cause Kaposi's sarcoma in patients with AIDS. In certain embodiments, the cancer is a cancer associated with EBV, HPV, HTLV-1, HTLV-2, or HHV-8. In certain embodiments, the cancer is Burkitt lymphoma, cervical cancer, genital cancer, adult T-cell leukemia/lymphoma, cutaneous T-cell lymphoma, or Kaposi's sarcoma.

In some embodiments, the cancer is a cancer other than a virus-associated cancer. In certain embodiments, the cancer is a cancer other than a cancer associated with EBV, HPV, HTLV-1, HTLV-2, or HHV-8. In certain embodiments, the cancer is a cancer other than Burkitt lymphoma, cervical cancer, genital cancer, adult T-cell leukemia/lymphoma, cutaneous T-cell lymphoma, or Kaposi's sarcoma. In one embodiment, the cancer is a tumor associated with Li-Fraumeni syndrome.

In some embodiments, the cancer is renal cell carcinoma, or kidney cancer, mesothelioma, hepatobilliary (hepatic and billiary duct), bone cancer, rhabdomyosarcoma, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, adrenocortical carcinoma, sarcoma of soft tissue, soft tissue and bone synovial sarcoma, cancer of the urethra, cancer of the penis, prostate cancer, testicular cancer, chronic or acute leukemia, chronic myeloid leukemia, acute myeloid leukemia, lymphocytic lymphomas, cancer of the bladder, cancer of the kidney or ureter, renal cell carcinoma, carcinoma of the renal pelvis, non-Hodgkin's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, squamous cell carcinoma of the head and neck (SCCHN); salivary gland cancer; neurofibromatosis-1 associated malignant peripheral nerve sheath tumors (MPNST); and medulloblastoma, or a combination of one or more of the foregoing cancers.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In certain embodiments, the cancer is a leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, or heavy chain disease. In one embodiment, the cancer is a solid tumor such as a sarcoma or carcinoma (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor.

### Subjects for Treatment

The above methods of treatment may be further characterized according to additional features, such as characteristics of the subject for treatment. For example, in certain embodiments, the subject has (i) expression of LINE1 RNA, LINE1 ORF1 polypeptide, and/or LINE1 ORF2 polypeptide; (ii) activity of LINE1 reverse transcriptase; (iii) expression of HERV-K RNA, and/or (iv) activity of HERV-K reverse transcriptase.

In certain embodiments, the subject has (i) expression of LINE1 RNA, LINE1 ORF1 polypeptide, and/or LINE1 ORF2 polypeptide; and/or (ii) activity of LINE1 reverse transcriptase. In certain embodiments, the subject has (i) elevated expression of LINE1 RNA, LINE1 ORF1 polypeptide, and/or LINE1 ORF2 polypeptide; and/or (ii) elevated activity of LINE1 reverse transcriptase. In certain embodiments, the subject has expression of LINE1 RNA, LINE1 ORF1 polypeptide, and/or LINE1 ORF2 polypeptide. In certain embodiments, the subject has expression of LINE1 RNA. In certain embodiments, the subject has expression of LINE1 ORF1 polypeptide. In certain embodiments, the subject has expression of LINE1 ORF2 polypeptide. In certain embodiments, the subject has activity of LINE1 reverse transcriptase.

In certain embodiments, the subject has (i) expression of HERV-K RNA, and/or (ii) activity of HERV-K reverse transcriptase. In certain embodiments, the subject has expression of HERV-K RNA. In certain embodiments, the subject has activity of HERV-K reverse transcriptase.

In certain embodiments, the subject has elevated (i) levels of LINE1 RNA, LINE1 ORF1 polypeptide, and/or LINE1 ORF2 polypeptide; (ii) activity of LINE1 reverse transcriptase; (iii) levels of HERV-K RNA, and/or (iv) activity of HERV-K reverse transcriptase.

In certain embodiments, the subject has elevated (i) levels of LINE1 RNA, LINE1 ORF1 polypeptide, and/or LINE1 ORF2 polypeptide; and/or (ii) activity of LINE1 reverse transcriptase. In certain embodiments, the subject has elevated levels of LINE1 RNA, LINE1 ORF1 polypeptide, and/or LINE1 ORF2 polypeptide. In certain embodiments, the subject has elevated levels of LINE1 RNA. In certain embodiments, the subject has elevated levels of LINE1 ORF1 polypeptide. In certain embodiments, the subject has elevated levels of LINE1 ORF2 polypeptide. In certain embodiments, the subject has elevated activity of LINE1 reverse transcriptase.

In certain embodiments, the subject has elevated (i) levels of HERV-K RNA, and/or (ii) activity of HERV-K reverse transcriptase. In certain embodiments, the subject has elevated levels of HERV-K RNA. In certain embodiments, the subject has elevated activity of HERV-K reverse transcriptase.

In certain embodiments, the subject is a human. In certain embodiments, the subject is an adult human. In certain embodiments, the subject is a pediatric human. In certain embodiments, the subject is a companion animal. In certain embodiments, the subject is a canine, feline, or equine.

### Uses of Compounds

Another aspect of the invention provides for the use of a compound described herein (such as a compound of Formula I, or other compounds in Section III) for treating a medical disorder, such as a medical disorder described herein.

Another aspect of the invention provides for the use of a compound described herein (such as a compound of Formula I, or other compounds in Section III) in the manufacture of a medicament. In certain embodiments, the medicament is for treating a disorder described herein, such as cancer.

I. Methods of Inhibiting LINE1 and/or HERV-K Reverse Transcriptase Activity in a Subject

Another aspect of the invention provides a method of inhibiting LINE1 reverse transcriptase activity in a subject suffering from a disorder selected from the group consisting of an autoimmune disorder and a neurological disorder. The method comprises contacting a LINE1 reverse transcriptase with an effective amount of a compound of Formula I, in order to inhibit the activity of said LINE1 reverse transcriptase; wherein Formula I is represented by:

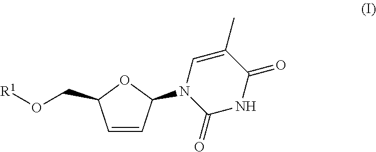

or a pharmaceutically acceptable salt thereof; wherein:
R¹ is

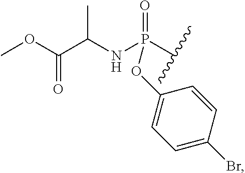 [SMILES: *P(=O)(NC(C)C(=O)OC)Oc1ccc(Br)cc1]

hydrogen, —P(O)(OH)₂, —P(O)(OH)—O—P(O)(OH)₂, or —(P(O)(OH))₂—O—P(O)(OH)₂.

In certain embodiments, the particular compound of Formula I is a compound defined by one of the embodiments described in Section III, below, such as

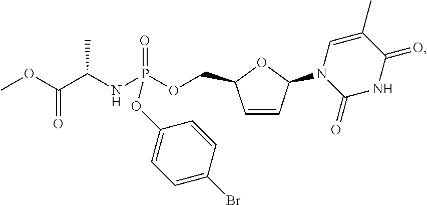 [SMILES: COC(=O)[C@H](C)NP(=O)(OC[C@@H]1C=C[C@H](n2cc(C)c(=O)[nH]c2=O)O1)Oc1ccc(Br)cc1]

or a pharmaceutically acceptable salt thereof. In certain embodiments, the disorder is a disorder defined by one of the embodiments described in Section I, above, such as an autoimmune disorder or a neurological disorder. In certain embodiments, the method further comprises inhibiting HERV-K reverse transcriptase activity in the subject.

Another aspect of the invention provides a method of inhibiting LINE1 reverse transcriptase activity in a subject suffering from a disorder that is cancer. The method comprises contacting a LINE1 reverse transcriptase with an effective amount of a compound of Formula I, in order to inhibit the activity of said LINE1 reverse transcriptase; wherein Formula I is represented by:

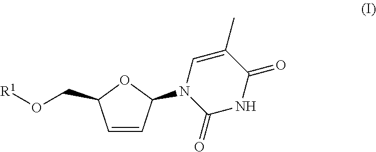

or a pharmaceutically acceptable salt thereof; wherein:
R¹ is

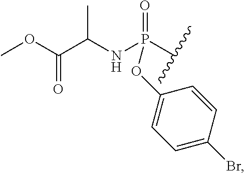 [SMILES: *P(=O)(NC(C)C(=O)OC)Oc1ccc(Br)cc1]

hydrogen, —P(O)(OH)₂, —P(O)(OH)—O—P(O)(OH)₂, or —(P(O)(OH))₂—O—P(O)(OH)₂.

In certain embodiments, the particular compound of Formula I is a compound defined by one of the embodiments described in Section III, below, such as

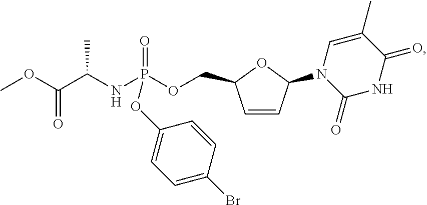 [SMILES: COC(=O)[C@H](C)NP(=O)(OC[C@@H]1C=C[C@H](n2cc(C)c(=O)[nH]c2=O)O1)Oc1ccc(Br)cc1]

or a pharmaceutically acceptable salt thereof. In certain embodiments, the disorder is a disorder defined by one of the embodiments described in Section I, above, such as a cancer that is a solid tumor or leukemia. In certain embodiments, the method further comprises inhibiting HERV-K reverse transcriptase activity in the subject.

Another aspect of the invention provides a method of inhibiting HERV-K reverse transcriptase activity in a subject suffering from a disorder selected from the group consisting of an autoimmune disorder and a neurological disorder. The method comprises contacting a HERV-K reverse transcriptase with an effective amount of a compound of Formula I, in order to inhibit the activity of said HERV-K reverse transcriptase; wherein Formula I is represented by:

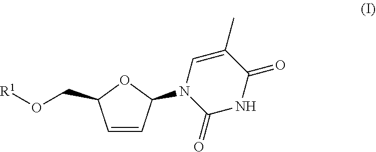

or a pharmaceutically acceptable salt thereof; wherein:
R¹ is

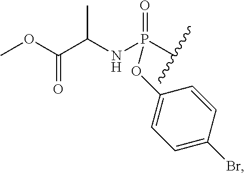 [SMILES: *P(=O)(NC(C)C(=O)OC)Oc1ccc(Br)cc1]

hydrogen, —P(O)(OH)₂, —P(O)(OH)—O—P(O)(OH)₂, or —(P(O)(OH))₂—O—P(O)(OH)₂.

In certain embodiments, the particular compound of Formula I is a compound defined by one of the embodiments described in Section III, below, such as

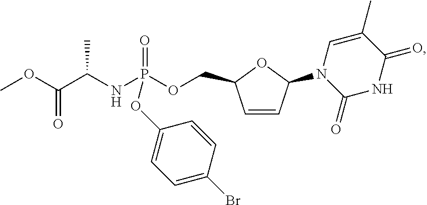 [SMILES: COC(=O)[C@H](C)NP(=O)(OC[C@@H]1C=C[C@H](n2cc(C)c(=O)[nH]c2=O)O1)Oc1ccc(Br)cc1]

or a pharmaceutically acceptable salt thereof. In certain embodiments, the disorder is a disorder defined by one of the embodiments described in Section I, above, such as an autoimmune disorder or a neurological disorder. In certain embodiments, the method further comprises inhibiting LINE1 reverse transcriptase activity in the subject.

Another aspect of the invention provides a method of inhibiting HERV-K reverse transcriptase activity in a subject suffering from a disorder that is cancer. The method comprises contacting a HERV-K reverse transcriptase with an effective amount of a compound of Formula I, in order to inhibit the activity of said HERV-K reverse transcriptase; wherein Formula I is represented by:

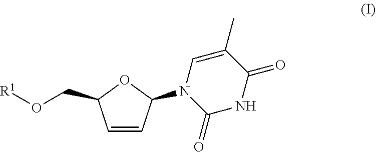

or a pharmaceutically acceptable salt thereof; wherein:
R¹ is

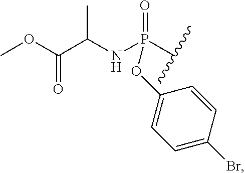 [SMILES: *P(=O)(NC(C)C(=O)OC)Oc1ccc(Br)cc1]

hydrogen, —P(O)(OH)₂, —P(O)(OH)—O—P(O)(OH)₂, or —(P(O)(OH))₂—O—P(O)(OH)₂.

In certain embodiments, the particular compound of Formula I is a compound defined by one of the embodiments described in Section III, below, such as

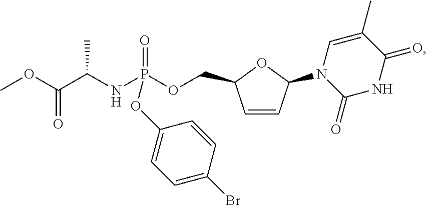 [SMILES: COC(=O)[C@H](C)NP(=O)(OC[C@@H]1C=C[C@H](n2cc(C)c(=O)[nH]c2=O)O1)Oc1ccc(Br)cc1]

or a pharmaceutically acceptable salt thereof. In certain embodiments, the disorder is a disorder defined by one of the embodiments described in Section I, above, such as a cancer that is a solid tumor or leukemia. In certain embodiments, the method further comprises inhibiting LINE1 reverse transcriptase activity in the subject.

Compounds may be tested for ability to inhibit LINE1 reverse transcriptase activity, for example, as described in the Examples. Compounds may be tested for ability to inhibit HERV-K reverse transcriptase activity, for example, as described in the Examples.

### III. Stampidine and Related Compounds

The methods described in Sections I and II above may be further characterized according to the compounds used in the methods. Exemplary compounds are described below, along with exemplary procedures for making the compounds.

In certain embodiments, the compound is a compound of Formula I represented by:

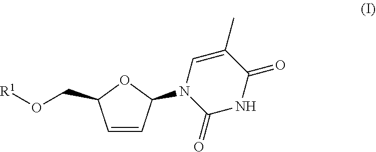

or a pharmaceutically acceptable salt thereof; wherein:
R¹ is

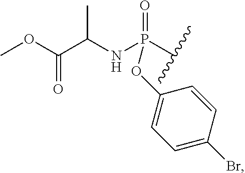 [SMILES: *P(=O)(NC(C)C(=O)OC)Oc1ccc(Br)cc1]

hydrogen, —P(O)(OH)₂, —P(O)(OH)—O—P(O)(OH)₂, or —(P(O)(OH))₂—O—P(O)(OH)₂.

In certain embodiments, the compound is a compound of Formula I, or a pharmaceutically acceptable salt thereof. In certain embodiments, the compound is a compound of Formula I.

In certain embodiments, in Formula I, R¹ is

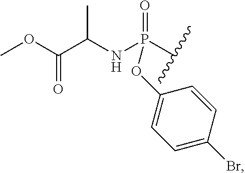 [SMILES: *P(=O)(NC(C)C(=O)OC)Oc1ccc(Br)cc1]

In certain embodiments, in Formula I, R¹ is

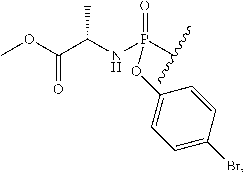 [SMILES: *P(=O)(N[C@@H](C)C(=O)OC)Oc1ccc(Br)cc1]

In certain embodiments, R¹ is hydrogen. In certain embodiments, R¹ is —P(O)(OH)₂, —P(O)(OH)—O—P(O)(OH)₂, or —(P(O)(OH))₂—O—P(O)(OH)₂.

In certain embodiments, the compound of Formula I is

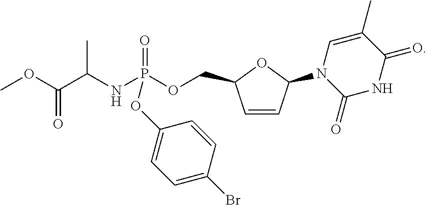 [SMILES: COC(=O)C(C)NP(=O)(OC[C@@H]1C=C[C@H](n2cc(C)c(=O)[nH]c2=O)O1)Oc1ccc(Br)cc1]

or a pharmaceutically acceptable salt thereof. In certain embodiments, the compound of Formula I is

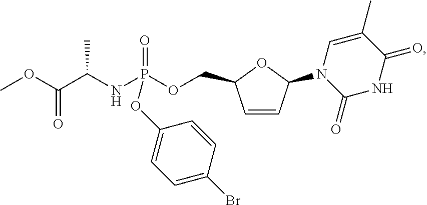 [SMILES: COC(=O)[C@H](C)NP(=O)(OC[C@@H]1C=C[C@H](n2cc(C)c(=O)[nH]c2=O)O1)Oc1ccc(Br)cc1]

or a pharmaceutically acceptable salt thereof. In certain embodiments, the compound of Formula I is

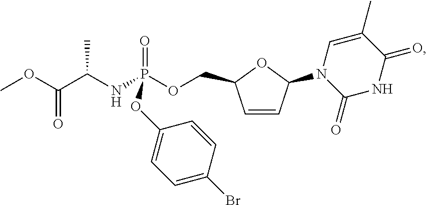 [SMILES: COC(=O)[C@H](C)N[P@](=O)(OC[C@@H]1C=C[C@H](n2cc(C)c(=O)[nH]c2=O)O1)Oc1ccc(Br)cc1]

or a pharmaceutically acceptable salt thereof. In certain embodiments, the compound of Formula I is

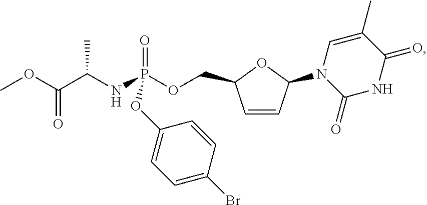 [SMILES: COC(=O)[C@H](C)N[P@@](=O)(OC[C@@H]1C=C[C@H](n2cc(C)c(=O)[nH]c2=O)O1)Oc1ccc(Br)cc1]

or a pharmaceutically acceptable salt thereof.

In certain embodiments, the compound of Formula I is

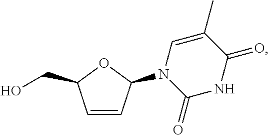 [SMILES: Cc1cn([C@H]2C=C[C@@H](CO)O2)c(=O)[nH]c1=O]

or a pharmaceutically acceptable salt thereof. In certain embodiments, the compound of Formula I is

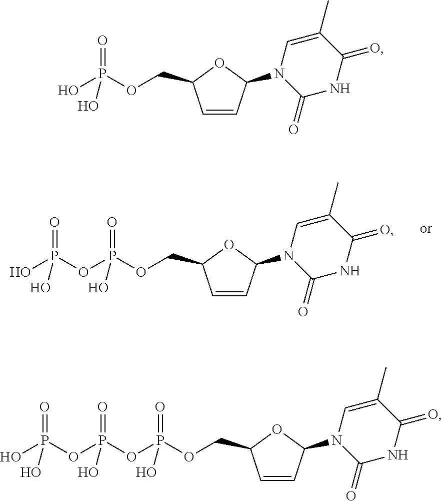 [SMILES: Cc1cn([C@H]2C=C[C@@H](COP(=O)(O)O)O2)c(=O)[nH]c1=O ; Cc1cn([C@H]2C=C[C@@H](COP(=O)(O)OP(=O)(O)O)O2)c(=O)[nH]c1=O ; "or" ; Cc1cn([C@H]2C=C[C@@H](COP(=O)(O)OP(=O)(O)OP(=O)(O)O)O2)c(=O)[nH]c1=O]

or a pharmaceutically acceptable salt thereof.

Methods for preparing stampidine, and related compounds of Formula I, are provided in, for example, international patent application WO 96/029336, U.S. Pat. No. 6,350,736, and related patents and applications, such as U.S. Pat. No. 6,455,513; and references therein. Additional methods and strategies which may be employed to provide phosphoramidate compounds, such as stampidine, include those described for preparing phosphoramidate compounds of 2′,3′-didehydro-3′-deoxy-4′-ethynylthymidine, provided in, for example, U.S. Pat. No. 8,022,083; WO 2006/110157; WO 2006/015261; and related patents and applications, such as U.S. Pat. Nos. 7,871,991; 8,318,701, and 8,329,926. Further strategies for preparing phosphoramidate compounds are described in, for example, Slusarczyk, M. et al. “Phosphoramidates and phosphonamidates (ProTides) with antiviral activity,” Antiviral Chemistry and Chemotherapy (2018), Vol. 26, p. 1-31, and references therein. Each of the foregoing references is hereby incorporated by reference in its entirety.

The modular synthetic routes described in the foregoing references can also be readily modified by one of skill in the art of organic synthesis to provide additional substituted stampidine and related compounds using strategies and reactions well known in the art, as described in, for example, “Comprehensive Organic Synthesis” (B. M. Trost & I. Fleming, eds., 1991-1992).

### IV. Combination Therapy

Another aspect of the invention provides for combination therapy. Stampidine or related compounds described herein (e.g., a compound of Formula I, or other compounds in Section III) or their pharmaceutically acceptable salts may be used in combination with additional therapeutic agents to treat medical disorders (e.g., according to the methods described in Section I, with disorders such as a cancer). Accordingly, in some embodiments, a method of the invention further comprises administering an effective amount of an additional therapeutic agent.

Each of the methods described herein for treating disease using combination therapy may be further characterized according to the additional therapeutic agent used in the method. For example, in certain embodiments, the additional therapeutic agent is tenofovir, a prodrug thereof, or a pharmaceutically acceptable salt of either of the foregoing. In certain embodiments, the additional therapeutic agent is tenofovir, tenofovir alafenamide, tenofovir amibufenamide, tenofovir disoproxil, or tenofovir exalidex; or a pharmaceutically acceptable salt thereof. In certain embodiments, the additional therapeutic agent is tenofovir, tenofovir alafenamide, tenofovir amibufenamide, tenofovir disoproxil, or tenofovir exalidex.

In certain embodiments, the additional therapeutic agent is tenofovir, or a pharmaceutically acceptable salt thereof. In certain embodiments, the additional therapeutic agent is tenofovir. In certain embodiments, the additional therapeutic agent is tenofovir alafenamide, or a pharmaceutically acceptable salt thereof. In certain embodiments, the additional therapeutic agent is tenofovir alafenamide. In certain embodiments, the additional therapeutic agent is tenofovir amibufenamide, or a pharmaceutically acceptable salt thereof. In certain embodiments, the additional therapeutic agent is tenofovir amibufenamide. In certain embodiments, the additional therapeutic agent is tenofovir disoproxil, or a pharmaceutically acceptable salt thereof. In certain embodiments, the additional therapeutic agent is tenofovir disoproxil, or a fumarate, succinate, maleate, orotate, aspartate, or phosphate salt thereof. In certain embodiments, the additional therapeutic agent is tenofovir disoproxil, or a fumarate, succinate, or maleate salt thereof. In certain embodiments, the additional therapeutic agent is tenofovir disoproxil. In certain embodiments, the additional therapeutic agent is tenofovir exalidex, or a pharmaceutically acceptable salt thereof. In certain embodiments, the additional therapeutic agent is tenofovir exalidex, or a potassium salt thereof. In certain embodiments, the additional therapeutic agent is tenofovir exalidex.

In some embodiments, the present invention provides a method of treating a disclosed disease or condition comprising administering to a patient in need thereof an effective amount of a compound disclosed herein or a pharmaceutically acceptable salt thereof and co-administering simultaneously, separately, or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially, or separately within a period of time from one another, for example within 1, 2, 3, 4, 5, 6, 7, 8, 9, 10, 11, 12, 13, 14, 15, 16, 17, 18, 19, 20, 21, 22, 23, or 24 hours from one another. In some embodiments, one or more other therapeutic agent and a compound or composition of the invention are administered as a multiple dosage regimen more than 24 hours apart.

The doses and dosage regimen of the active ingredients used in the combination therapy may be determined by an attending clinician. In certain embodiments, the stampidine or related compound described herein (e.g., a compound of Formula I, or other compounds in Section III) and the additional therapeutic agent(s) (e.g. the second, third, or fourth, or fifth anti-cancer agent, described below) are administered in doses commonly employed when such agents are used as monotherapy for treating the disorder. In other embodiments, the stampidine or related compound described herein (e.g., a compound of Formula I, or other compounds in Section III) and the additional therapeutic agent(s) (e.g. the second, third, or fourth, or fifth anti-cancer agent, described below) are administered in doses lower than the doses commonly employed when such agents are used as monotherapy for treating the disorder. In certain embodiments, the stampidine or related compound described herein (e.g., a compound of Formula I, or other compounds in Section III) and the additional therapeutic agent(s) (e.g. the second, third, or fourth, or fifth anti-cancer agent, described below) are present in the same composition, which is suitable for oral administration.

In certain embodiments, the stampidine or related compound described herein (e.g., a compound of Formula I, or other compounds in Section III) and the additional therapeutic agent(s) (e.g. the second, third, or fourth, or fifth anti-cancer agent, described below) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

Another aspect of this invention is a kit comprising a therapeutically effective amount of stampidine or related compound described herein (e.g., a compound of Formula I, or other compounds in Section III), a pharmaceutically acceptable carrier, vehicle or diluent, and optionally at least one additional therapeutic agent listed above.

### Cancer

Accordingly, another aspect of the invention provides a method of treating cancer in a patient. The method comprises administering to a subject in need thereof (i) a therapeutically effective amount of stampidine or related compound described herein and (ii) a second anti-cancer agent, in order to treat the cancer.

In certain embodiments, the second anti-cancer agent is radiation therapy.

In certain embodiments, the second anti-cancer agent is a therapeutic antibody. In certain embodiments, the therapeutic antibody targets one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, interleukin-1 beta, B4GALNT1, interleukin-6, and interleukin-6 receptor. In certain embodiments, the therapeutic antibody targets VEGFR3 or TRAILR3.

In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, CDP791, ramucirumab, etaracizumab, volociximab, cetuximab, panitumumab, nimotuzumab, 806, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 81C6, MEDI551, lirilumab, MEDI9447, daratumumab, belimumab, canakinumab, dinutuximab, siltuximab, and tocilizumab.

In certain embodiments, the second anti-cancer agent is a cytokine. In certain embodiments, the cytokine is IL-12, IL-15, GM-CSF, or G-CSF.

In certain embodiments, the second anti-cancer agent is sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), or 2-chloro-deoxyadenosine. In certain embodiments, the second anti-cancer agent is pazopanib, sorafenib, axitinib, lenvatinib, afatinib, erlotinib, gefitinib, osimertinib, or vandetanib.

In certain embodiments, the second anti-cancer agent is a placental growth factor, an antibody-drug conjugate, an oncolytic virus, or an anti-cancer vaccine. In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is an immune checkpoint inhibitor (also referred to as immune checkpoint blockers). Immune checkpoint inhibitors are a class of therapeutic agents that have the effect of blocking immune checkpoints. See, for example, Pardoll in Nature Reviews Cancer (2012) vol. 12, pages 252-264. In certain embodiments, the immune checkpoint inhibitor is an agent that inhibits one or more of (i) cytotoxic T-lymphocyte-associated antigen 4 (CTLA4), (ii) programmed cell death protein 1 (PD1), (iii) PDL1, (iv) LAB3, (v) B7-H3, (vi) B7-H4, and (vii) TIM3. In certain embodiments, the immune checkpoint inhibitor is ipilumumab. In certain embodiments, the immune checkpoint inhibitor is pembrolizumab. In certain embodiments, the immune checkpoint inhibitor is atezolizumab, cemiplimab, cemiplimab-rwlc, dostarlimab, durvalumab, or nivolumab.

In certain embodiments, the second anti-cancer agent is a monoclonal antibody that targets a non-checkpoint target (e.g., Herceptin). In certain embodiments, the second anti-cancer agent is a non-cytoxic agent (e.g., a kinase inhibitor).

In certain embodiments, the second anti-cancer agent is selected from mitomycin, ribomustin, vincristine, tretinoin, etoposide, cladribine, gemcitabine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, aminoglutethimide, amsacrine, proglumide, elliptinium acetate, ketanserin, doxifluridine, etretinate, isotretinoin, streptozocin, nimustine, vindesine, cytarabine, bicalutamide, vinorelbine, vesnarinone, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, colony stimulating factor-1, colony stimulating factor-2, denileukin diftitox, interleukin-2, leutinizing hormone releasing factor, interferon-alpha, interferon-2 alpha, interferon-beta, interferon-gamma.

In certain embodiments, the second anti-cancer agent is paclitaxel, docetaxel, cisplatin, epirubicin, 5-fluorouracil, or capecitabine. In certain embodiments, the second anti-cancer agent is ixabepilone or eribulin. In certain embodiments, the second anti-cancer agent is ART558. In certain embodiments, the second anti-cancer agent is lapatinib, neratinib, or tucatinib. In certain embodiments, the second anti-cancer agent is fulvestrant. In certain embodiments, the second anti-cancer agent is anastrozole or exemestane. In certain embodiments, the second anti-cancer agent is MK2206. In certain embodiments, the second anti-cancer agent is dacomitinib, mobocertinib, necitumumab, or amivantamab. In certain embodiments, the second anti-cancer agent is pemetrexed. In certain embodiments, the second anti-cancer agent is brigatinib. In certain embodiments, the second anti-cancer agent is capmatinib or tepotinib. In certain embodiments, the second anti-cancer agent is entrectinib. In certain embodiments, the second anti-cancer agent is pralsetinib or selpercatinib. In certain embodiments, the second anti-cancer agent is ipilimumab. In certain embodiments, the second anti-cancer agent is sotorasib. In certain embodiments, the second anti-cancer agent is topotecan or irinotecan. In certain embodiments, the second anti-cancer agent is lurbinectedin, melphalan, or thiotepa. In certain embodiments, the second anti-cancer agent is trifluridine or tipiracil. In certain embodiments, the second anti-cancer agent is megestrol. In certain embodiments, the second anti-cancer agent is sunitinib. In certain embodiments, the second anti-cancer agent is lanreotide or lutetium. In certain embodiments, the second anti-cancer agent is belzutifan.

In certain embodiments, the second anti-cancer agent is an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, or a WEE1 Inhibitor.

In certain embodiments, the second anti-cancer agent is an ALK Inhibitor. In certain embodiments, the second anti-cancer agent is an ALK Inhibitor comprising ceritinib or crizotinib. In certain embodiments, the second anti-cancer agent is an ALK Inhibitor comprising lorlatinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an ATR Inhibitor comprising BAY1895344 or M4344. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a CHK1 Inhibitor comprising AZD7762. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a Cyclin-Dependent Kinase Inhibitor comprising abemaciclib or ribociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparib, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is a Phosphoinositide 3-Kinase Inhibitor comprising alpelisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is an agonist of OX40, CD137, CD40, or GITR. In certain embodiments, the second anti-cancer agent is an agonist of CD27, HVEM, TNFRSF25, or ICOS.

In certain embodiments, the second anti-cancer agent is tenofovir, a prodrug thereof, or a pharmaceutically acceptable salt of either of the foregoing. In certain embodiments, the second anti-cancer agent is tenofovir, tenofovir alafenamide, tenofovir amibufenamide, tenofovir disoproxil, or tenofovir exalidex; or a pharmaceutically acceptable salt thereof. In certain embodiments, the second anti-cancer agent is tenofovir, tenofovir alafenamide, tenofovir amibufenamide, tenofovir disoproxil, or tenofovir exalidex.

In certain embodiments, the second anti-cancer agent is tenofovir, or a pharmaceutically acceptable salt thereof. In certain embodiments, the second anti-cancer agent is tenofovir. In certain embodiments, the second anti-cancer agent is tenofovir alafenamide, or a pharmaceutically acceptable salt thereof. In certain embodiments, the second anti-cancer agent is tenofovir alafenamide. In certain embodiments, the second anti-cancer agent is tenofovir amibufenamide, or a pharmaceutically acceptable salt thereof. In certain embodiments, the second anti-cancer agent is tenofovir amibufenamide. In certain embodiments, the second anti-cancer agent is tenofovir disoproxil, or a pharmaceutically acceptable salt thereof. In certain embodiments, the second anti-cancer agent is tenofovir disoproxil, or a fumarate, succinate, maleate, orotate, aspartate, or phosphate salt thereof. In certain embodiments, the second anti-cancer agent is tenofovir disoproxil, or a fumarate, succinate, or maleate salt thereof. In certain embodiments, the second anti-cancer agent is tenofovir disoproxil. In certain embodiments, the second anti-cancer agent is tenofovir exalidex, or a pharmaceutically acceptable salt thereof. In certain embodiments, the second anti-cancer agent is tenofovir exalidex, or a potassium salt thereof. In certain embodiments, the second anti-cancer agent is tenofovir exalidex.

In certain embodiments, the method further comprises administering to the subject a third anti-cancer agent. In certain embodiments, the method further comprises administering to the subject a fourth anti-cancer agent. In certain embodiments, the method further comprises administering to the subject a fifth anti-cancer agent.

In certain embodiments, the third anti-cancer agent is one of the second anti-cancer agents described above. In certain embodiments, the fourth anti-cancer agent is one of the second anti-cancer agents described above. In certain embodiments, the fifth anti-cancer agent is one of the second anti-cancer agents described above.

### Autoimmune Disorders

Another aspect of the invention provides a method of treating an autoimmune disorder in a patient. The method comprises administering to a subject in need thereof (i) a therapeutically effective amount of stampidine or a related compound described herein and (ii) a second therapeutic agent, in order to treat the autoimmune disorder.

In certain embodiments, the second therapeutic agent is a small molecule or a recombinant biologic agent. In certain embodiments, the second therapeutic agent is selected from acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®, Neoral®), tacrolimus, sirolimus, mycophenolate, leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), anti-T cell antibodies such as Thymoglobulin, IV Immunoglobulins (IVIg), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), anti-IL36 agents such as BI655130, Dihydroorotate dehydrogenase inhibitors such as IMU-838, anti-OX40 agents such as KHK-4083, microbiome agents such as RBX2660, SER-287, Narrow spectrum kinase inhibitors such as TOP-1288, anti-CD40 agents such as BI-655064 and FFP-104, guanylate cyclase agonists such as dolcanatide, sphingosine kinase inhibitors such as opaganib, anti-IL-12/IL-23 agents such as AK-101, Ubiquitin protein ligase complex inhibitors such as BBT-401, sphingosine receptors modulators such as BMS-986166, P38MAPK/PDE4 inhibitors such as CBS-3595, CCR9 antagonists such as CCX-507, FimH antagonists such as EB-8018, HIF-PH inhibitors such as FG-6874, HIF-1a stabilizer such as GB-004, MAP3K8 protein inhibitors such as GS-4875, LAG-3 antibodies such as GSK-2831781, RIP2 kinase inhibitors such as GSK-2983559, Farnesoid X receptor agonist such as MET-409, CCK2 antagonists such as PNB-001, IL-23 Receptor antagonists such as PTG-200, Purinergic P2X7 receptor antagonists such as SGM-1019, PDE4 inhibitors such as Apremilast, ICAM-1 inhibitors such as alicaforsen sodium, Anti-IL23 agents such as guselkumab, brazikumab and mirkizumab, ant-IL-15 agents such as AMG-714, TYK-2 inhibitors such as BMS-986165, NK Cells activators such as CNDO-201, RIP-1 kinase inhibitors such as GSK-2982772, anti-NKGD2 agents such as JNJ-4500, CXCL-10 antibodies such as JT-02, IL-22 receptor agonists such as RG-7880, GATA-3 antagonists such as SB-012, and Colony-stimulating factor-1 receptor inhibitors such as edicotinib.

In certain embodiments, the second therapeutic agent is pentoxifylline, propentofylline, torbafylline, cyclosporine, methotrexate, tamoxifen, forskolin and analogs thereof, tar derivatives, steroids, vitamin A and its derivatives, vitamin D and its derivatives, a cytokine, a chemokine, a stem cell growth factor, a lymphotoxin, an hematopoietic factor, a colony stimulating factor (CSF), erythropoietin, thrombopoietin, tumor necrosis factor-α (TNF), TNF-Θ, granulocyte-colony stimulating factor (G-CSF), granulocyte macrophage-colony stimulating factor (GM-CSF), interferon-α, interferon-β, interferon-γ, interferon-λ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), IL-1, IL-1a, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, or lymphotoxin.

In certain embodiments, the second therapeutic agent is tenofovir, a prodrug thereof, or a pharmaceutically acceptable salt of either of the foregoing. In certain embodiments, the second therapeutic agent is tenofovir, tenofovir alafenamide, tenofovir amibufenamide, tenofovir disoproxil, or tenofovir exalidex; or a pharmaceutically acceptable salt thereof. In certain embodiments, the second therapeutic agent is tenofovir, tenofovir alafenamide, tenofovir amibufenamide, tenofovir disoproxil, or tenofovir exalidex.

In certain embodiments, the second therapeutic agent is tenofovir, or a pharmaceutically acceptable salt thereof. In certain embodiments, the second therapeutic agent is tenofovir. In certain embodiments, the second therapeutic agent is tenofovir alafenamide, or a pharmaceutically acceptable salt thereof. In certain embodiments, the second therapeutic agent is tenofovir alafenamide. In certain embodiments, the second therapeutic agent is tenofovir amibufenamide, or a pharmaceutically acceptable salt thereof. In certain embodiments, the second therapeutic agent is tenofovir amibufenamide. In certain embodiments, the second therapeutic agent is tenofovir disoproxil, or a pharmaceutically acceptable salt thereof. In certain embodiments, the second therapeutic agent is tenofovir disoproxil, or a fumarate, succinate, maleate, orotate, aspartate, or phosphate salt thereof. In certain embodiments, the second therapeutic agent is tenofovir disoproxil, or a fumarate, succinate, or maleate salt thereof. In certain embodiments, the second therapeutic agent is tenofovir disoproxil. In certain embodiments, the second therapeutic agent is tenofovir exalidex, or a pharmaceutically acceptable salt thereof. In certain embodiments, the second therapeutic agent is tenofovir exalidex, or a potassium salt thereof. In certain embodiments, the second therapeutic agent is tenofovir exalidex.

In certain embodiments, the method further comprises administering to the subject a third therapeutic agent. In certain embodiments, the method further comprises administering to the subject a fourth therapeutic agent. In certain embodiments, the method further comprises administering to the subject a fifth therapeutic agent.

In certain embodiments, the third therapeutic agent is one of the second therapeutic agents described above. In certain embodiments, the fourth therapeutic agent is one of the second therapeutic agents described above. In certain embodiments, the fifth therapeutic agent is one of the second therapeutic agents described above.

### Neurological Disorders

Another aspect of the invention provides a method of treating a neurological disorder in a patient. The method comprises administering to a subject in need thereof (i) a therapeutically effective amount of stampidine or a related compound described herein and (ii) a second therapeutic agent, in order to treat the neurological disorder.

In certain embodiments, the second therapeutic agent is a dopaminergic treatment, a cholinesterase inhibitor, an antipsychotic drug, deep brain stimulation (for example, to stop tremor and refractory movement disorders), riluzole, a caffein A2A receptor antagonist, pramipexole, or rasagilin.

In certain embodiments, the second therapeutic agent is tenofovir, a prodrug thereof, or a pharmaceutically acceptable salt of either of the foregoing. In certain embodiments, the second therapeutic agent is tenofovir, tenofovir alafenamide, tenofovir amibufenamide, tenofovir disoproxil, or tenofovir exalidex; or a pharmaceutically acceptable salt thereof. In certain embodiments, the second therapeutic agent is tenofovir, tenofovir alafenamide, tenofovir amibufenamide, tenofovir disoproxil, or tenofovir exalidex.

In certain embodiments, the second therapeutic agent is tenofovir, or a pharmaceutically acceptable salt thereof. In certain embodiments, the second therapeutic agent is tenofovir. In certain embodiments, the second therapeutic agent is tenofovir alafenamide, or a pharmaceutically acceptable salt thereof. In certain embodiments, the second therapeutic agent is tenofovir alafenamide. In certain embodiments, the second therapeutic agent is tenofovir amibufenamide, or a pharmaceutically acceptable salt thereof. In certain embodiments, the second therapeutic agent is tenofovir amibufenamide. In certain embodiments, the second therapeutic agent is tenofovir disoproxil, or a pharmaceutically acceptable salt thereof. In certain embodiments, the second therapeutic agent is tenofovir disoproxil, or a fumarate, succinate, maleate, orotate, aspartate, or phosphate salt thereof. In certain embodiments, the second therapeutic agent is tenofovir disoproxil, or a fumarate, succinate, or maleate salt thereof. In certain embodiments, the second therapeutic agent is tenofovir disoproxil. In certain embodiments, the second therapeutic agent is tenofovir exalidex, or a pharmaceutically acceptable salt thereof. In certain embodiments, the second therapeutic agent is tenofovir exalidex, or a potassium salt thereof. In certain embodiments, the second therapeutic agent is tenofovir exalidex.

In certain embodiments, the method further comprises administering to the subject a third therapeutic agent. In certain embodiments, the method further comprises administering to the subject a fourth therapeutic agent. In certain embodiments, the method further comprises administering to the subject a fifth therapeutic agent.

In certain embodiments, the third therapeutic agent is one of the second therapeutic agents described above. In certain embodiments, the fourth therapeutic agent is one of the second therapeutic agents described above. In certain embodiments, the fifth therapeutic agent is one of the second therapeutic agents described above.

### V. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carrier, adjuvant, and/or vehicle. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

In certain embodiments, the invention provides a pharmaceutical composition comprising a compound described herein (e.g., a compound of Formula I) and a pharmaceutically acceptable carrier. In certain embodiments, the invention provides a pharmaceutical composition comprising a compound described herein (e.g., a compound of Formula I), an additional therapeutic agent (e.g., a compound described in Section IV), and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, troches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising stampidine or a related compound described herein in a therapeutically effective amount for the treatment of a medical disorder described herein.

### EXAMPLES

The invention now being generally described, will be more readily understood by reference to the following examples, which are included merely for purposes of illustration of certain aspects and embodiments of the present invention, and are not intended to limit the invention. Starting materials described herein can be obtained from commercial sources or may be readily prepared from commercially available materials using transformations known to those of skill in the art.

### Example 1—Transient Cellular Assay for Inhibiting LINE1 Reverse Transcriptase

An exemplary compound was tested for ability to inhibit LINE1 reverse transcriptase using a transient artificial-intron Cis LINE1 reporter assay. Assay procedures and results are described below.

### Part I—Procedure for Transient Artificial-Intron Cis LINE1 Reporter Assay

Intron-disrupted Firefly luciferase (FLuc) expression cassettes were generated as described by Xie, Y. et al. “Characterization of L1 retrotransposition with high-throughput dual-luciferase assays,” Nucleic Acid Res. (2011) Vol. 39, No. 3, e16. In addition, the plasmid contained an intact Renilla luciferase (RLuc) expression cassette on the vector backbone, in order to normalize transfection efficiency and measure potential cell toxicity.

HEK 293 cells were seeded in 96-well plates at 1,000 cells/well in 55 μL and grown for 24 hours. Cells were transfected with FuGeneHD (Promega) following the manufacturer's protocol. Each well received 0.133 ng plasmid, 0.4 μL FuGeneHD reagent, and 4.5 μL GlutaMAX-I-supplemented Opti-MEM I reduced-serum medium (Invitrogen). Cells were simultaneously treated with test compound serially diluted starting at 100 μM in a 3-fold dilution dose response.

Luminescence was measured using the Dual-Glo Luciferase Assay System (Promega) following the manufacturer's instructions. The ratio between FLuc and RLuc gene expression was used to report LINE1 activity.

### Part II—Results

Experimental results are provided in FIG. 1, where the following test compound was found to inhibit FLuc (labeled “FF/RR” in FIG. 1) with an IC₅₀ of 5.7 nM:

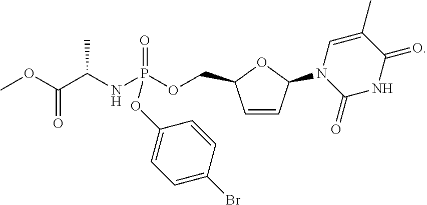 [SMILES: COC(=O)[C@H](C)NP(=O)(OC[C@@H]1C=C[C@H](n2cc(C)c(=O)[nH]c2=O)O1)Oc1ccc(Br)cc1]

### Example 2—Stable Cellular Assay for Inhibiting LINE1 Reverse Transcriptase

An exemplary compound was tested for ability to inhibit LINE1 reverse transcriptase using a stable artificial-intron Cis LINE1 reporter assay. Assay procedures and results are described below.

### Part I—Procedure for Stable Artificial-Intron Cis LINE1 Reporter Assay

A stable HeLa Tet-On 3G (Takara, cat no 631183) cell line expressing a bi-directional inducible LINE1 construct was generated as described in Xie, Y. et al. “Cell division promotes efficient retrotransposition in a stable L1 reporter cell line,” Mobile DNA (2013) 4:10. Single cell clones were screened for high Luciferase expression and the highest expression Firefly expressing clone was chosen for compound testing.

Test compound was serially diluted in DMSO and spotted in 96-well plates. Subsequently the HeLa L1 artificial-intron reporter cells were plated into the compound-containing wells (8,000 cells/well), and the cells were induced for reporter expression with doxycycline (Sigma cat no D9891) at a final concentration of 500 ng/mL. Luminescence was measured 72 h after plating using the Dual-Glo Luciferase Assay System (Promega cat no E2940) following the manufacturer's instructions. The Firefly Luciferase activity was used to report LINE1 activity.

### Part II—Results

The following test compound was found to inhibit LINE1 reverse transcriptase with an IC₅₀ of 0.017 μM:

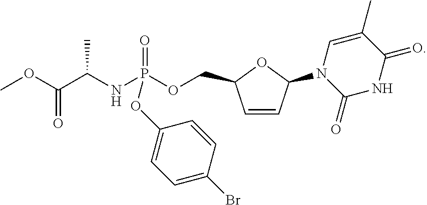 [SMILES: COC(=O)[C@H](C)NP(=O)(OC[C@@H]1C=C[C@H](n2cc(C)c(=O)[nH]c2=O)O1)Oc1ccc(Br)cc1]

### Example 3—Biochemical Assay for Inhibiting LINE1 Reverse Transcriptase

An exemplary compound was tested for ability to inhibit LINE1 reverse transcriptase using a homogeneous time-resolved fluorescence (HTRF) assay. Assay procedures and results are described below.

### Part I—Procedure for Homogeneous Time-Resolved Fluorescence LINE1 RT Assay

The LINE1 reverse transcriptase homogeneous time-resolved fluorescence (HTRF) assay was performed with recombinant MBP-tagged LINE1 protein (238-1061) (generated and purified according to procedures in Dai L. et al. BMC Biochemistry 2011; 12:18) in a 384-well format. Test compound was serially diluted in DMSO and further diluted in the assay buffer (50 mM Tris-HCl, 50 mM KCl, 10 mM MgCl₂, 10 mM DTT, pH 8.1) to achieve a final DMSO concentration of 1%. The serially diluted compound was mixed with 64 ng/well of LINE1 enzyme, 5 nM of pre-annealed template/biotin-primer pair (synthesized at Generay Biotechnology), 10 nM of Fluorescein-12-dATP fluorescent probe (Perkin Elmer), and 1 μM dGTP/dCTP/dTTP (Thermo Fisher Scientific) in the assay buffer. The template/biotin-primer sequences were as follows:

(SEQ ID NO: 1)
5′ to 3′ GUAACUAGAGAUCCCUCAGACCCUUUUAGUCAGAAU
(SEQ ID NO: 2)
5′ to 3′ Biotin-TTCTGACTAAAAGGGTCTGAGGGAT.

After incubating at 25° C. for 60 minutes, the detection reagent (20 mM EDTA with streptavidin-terbium cryptate, Cisbio Bioassay) in the PPI buffer (Cisbio Bioassay) was added, and the mixture was incubated at 25° C. for 30 minutes. At the end of the incubation, fluorescence was read at ex/em=337/485 nm and ex/em=337/520 nm on an Envision 2104 plate reader (Perkin Elmer). The fluorescence ratio at 520/485 nm was used for the calculation. Percent inhibition was calculated with the DMSO sample as 0% inhibition and no enzyme as 100% inhibition. The IC₅₀ was calculated by fitting the compound dose inhibition curve with a 4-parameter non-linear regression equation.

### Part II—Results

The trisodium salt of the following compound was found to inhibit LINE1 reverse transcriptase with an IC₅₀ of 23 nM:

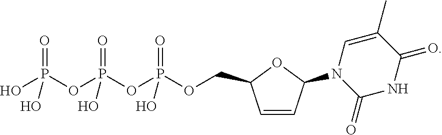 [SMILES: Cc1cn([C@H]2C=C[C@@H](COP(=O)(O)OP(=O)(O)OP(=O)(O)O)O2)c(=O)[nH]c1=O]

### Example 4—Biochemical Assay for Inhibiting HERV-K Reverse Transcriptase

An exemplary compound was tested for ability to inhibit HERV-K reverse transcriptase using a homogeneous time-resolved fluorescence (HTRF) assay. Assay procedures and results are described below.

### Part I—Procedure for Homogeneous Time-Resolved Fluorescence HERV-K RT Assay

The HERV-K reverse transcriptase homogeneous time-resolved fluorescence (HTRF) assay was performed in a 384-well format with HERV-K reverse transcriptase (2-596)-8His protein. Baculoviruses were created using Bac-to-Bac technology (Invitrogen). pFastBac donor plasmids containing HERV-K reverse transcriptase sequence (NCBI GenBank number AAC63291.1, J. Virology (1999) Vol. 73, No. 3, pp. 2365-2375) were transformed into DH10 Bac cells following the manufacturer's instructions. Recombinant bacmids were then isolated clonally and transfected into SF9 cells with lipofectin. HERV-K reverse transcriptase was expressed in the SF9 insect cells and then purified using immobilized metal affinity chromatography (IMAC) followed by size-exclusion chromatography (SEC).

Test compound was serially diluted in DMSO and further diluted in the assay buffer (50 mM Tris-HCl, 50 mM KCl, 10 mM MgCl₂, 10 mM DTT, pH 8.1) to achieve a final DMSO concentration of 1%. The serially diluted compound was mixed with 16 ng/well of HERV-K enzyme, 10 nM of pre-annealed template/biotin-primer pair (synthesized at Geneway Biotechnology), and 10 nM of Fluorescein-12-dUTP fluorescent probe (Perkin Elmer). The template/biotin-primer sequences were poly(rA₄₅) and biotin-oligo(dT)₁₆.

After incubating at 25° C. for 30 minutes, the detection reagent 20 mM EDTA with streptavidin-terbium cryptate (Cisbio Bioassay) in the PPI buffer (Cisbio Bioassay) was added, and the mixture was incubated at 25° C. for 30 minutes. At the end of the incubation, fluorescence was read at ex/em=337/485 nm and ex/em=337/520 nm on an Envision 2104 plate reader (Perkin Elmer). The fluorescence ratio at 520/485 nm was used for the calculation. Percent inhibition was calculated with the DMSO sample as 0% inhibition and no enzyme as 100% inhibition. The IC₅₀ was calculated by fitting the compound dose inhibition curve with a 4-parameter non-linear regression equation.

### Part II—Results

The trisodium salt of the following compound was found to inhibit HERV-K reverse transcriptase with an IC₅₀ of 42 nM:

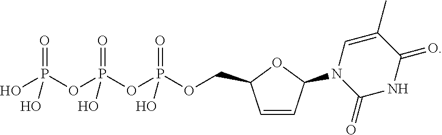 [SMILES: Cc1cn([C@H]2C=C[C@@H](COP(=O)(O)OP(=O)(O)OP(=O)(O)O)O2)c(=O)[nH]c1=O]

### Example 5—In Vivo Decitabine Challenge Model

Twenty 9-11 week old C₅₇BL/6 mice were acclimated to the lab for at least 5 days. Stampidine was prepared in a 20% HP-b-CD in water solution for i.p. administration. Decitabine (Sigma) was dissolved in sterile PBS (pH 7.4) and dosed within 30 minutes of preparation of the solution. Doses of both stampidine and decitabine were administered i.p. once a day, every day from Day 0 to Day 4.

On Day 0, mice were split into four groups of five mice and given their first dose of decitabine (i.p., 5 mg/kg) and stampidine. Dosing groups were:

| Group | Stampidine Dose (mg/kg) |
|---|---|
| Control | 0 |
| 1 | 3 |
| 2 | 15 |
| 3 | 75 |

Decitabine and stampidine were administered daily from Day 0 to Day 4. All mice were euthanized 1 hour after the last dose administration on Day 4. Spleens, liver, and terminal colon were collected, along with plasma from each animal. The fold changes in interferon-stimulated gene (ISG) expression were calculated by first normalizing to GAPDH gene using the Delta CT method. The CT (gene of interest)−CT (reference gene) was calculated to generate a delta CT for all samples. The fold change was then calculated by taking the Log2(Delta CT(control)−Delta CT (experimental). The control in this example was the PBS control animal group. The Taqman duplex assay was used to determine levels of GAPDH v. IFIT2.

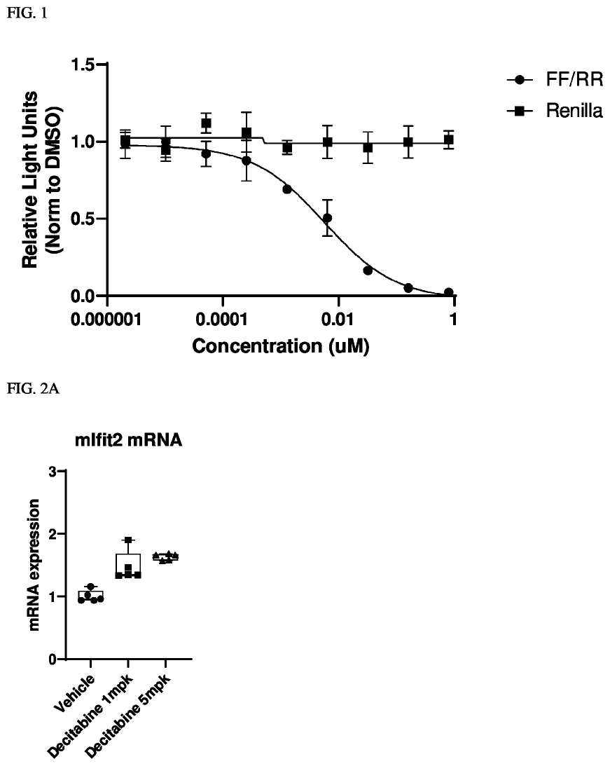

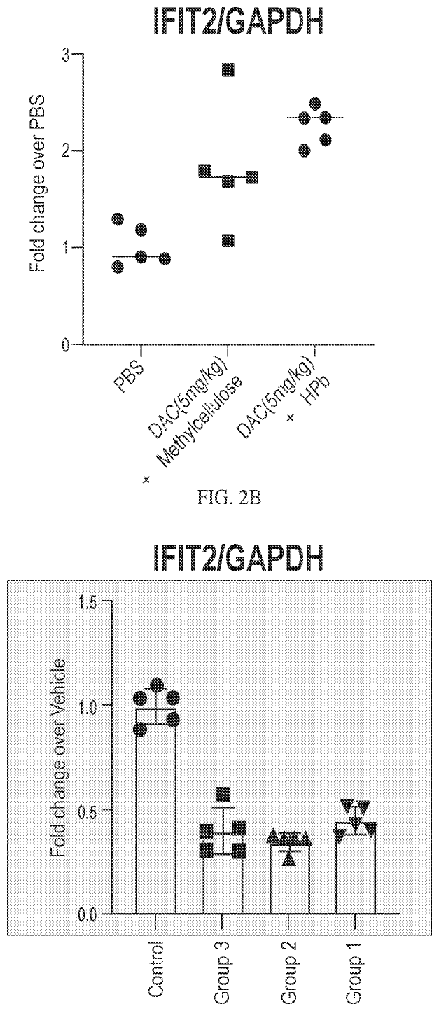

Repeated dosing of decitabine induced interferon-stimulated gene (ISG) response in the spleen in the control animals (see FIG. 2A and FIG. 2B). Dosing of stampidine showed the change in ISG response which was most prominent in the spleen tissue (see FIG. 3).

### Example 6—Decitabine-Stimulated Human PBMC Assay

EasySep buffer (32 mL, Stem Cell, cat. #20144) was used to dilute 8 mL of LRSC buffy coat (from fresh Leukopak) with gentle mixing. The diluted buffy coat (20 mL) was transferred into each of two SepMate 50 tubes, and the tubes were filled with 15 mL of Lyphoprep (Stem Cell, ct. #07851) density gradient. The SepMate tubes were then centrifuged at 1200G for 10 minutes at room temperature with the brake on. The top layer of supernatant was collected in SepMate tubes by quickly pouring it into a new 50 mL conical tube. The PBMCs were washed with EasySep buffer×2 by centrifuging at 300G for 5 minutes. The cells were resuspended in 30 mL of EasySep and centrifuged at 100G for 5 minutes with the brake off, and the platelets were removed. The cells were then resuspended in 6 mL of 1× RBC lysis buffer (InvitroGen) and incubated at 37° C. for 5 minutes. Then, 25 mL of EasySep buffer was mixed into the tube and it was centrifuged at 300G for 5 minutes. The cells were resuspended in 10 mL of EasySep buffer and the cells were then counted with Cellometer (AO/PI). The PBMCs were resuspended in RPMI1640 (ThermoFisher)+10% FBS (HyClone)+p/s at 3×10⁶/mL. The PBMCs (100 μL, 300 k PBMCs) were then seeded in a 96-well flat bottom microplate (Corning) that had been precoated with 100 μL of anti-CD3 antibody (10 μg/mL in PBS, Biolegend) or PBS at 4° C., one day before the assay was commenced.

To each well, the following solutions were added: 1) 100 μL of cells (final cell number per well is 3×10⁵ cell/well); 2) 25 μL of anti-CD28 antibody at 6× (5 μg/mL final concentration, Biolegend); 3) 25 μL of decitabine at 6× (10 μM final concentration); and 4) the Compound in DMSO was dispensed directly into each well with a d300e digital dispense (Tecan). The final concentration of DMSO for each well was normalized to 0.3%. The plate was incubated at 37° C. without any agitation for 5 days. Samples were collected 120 hours after incubation to determine IFN-β and IL-2 levels using a U-PLEX Human IFNb Assay Sector (5PL) (MSD, cat. #K151VIK-2).

After 5 days, the plate was spun down at 100×G for 5 minutes. Supernatants (100 μL) was collected for interferon β (IFN-β) analysis using the MSD assay noted above, and any residual supernatant was stored at −80° C. Cell viability was checked to determine if cell death had an impact on the IFN-β levels detected.

### Results

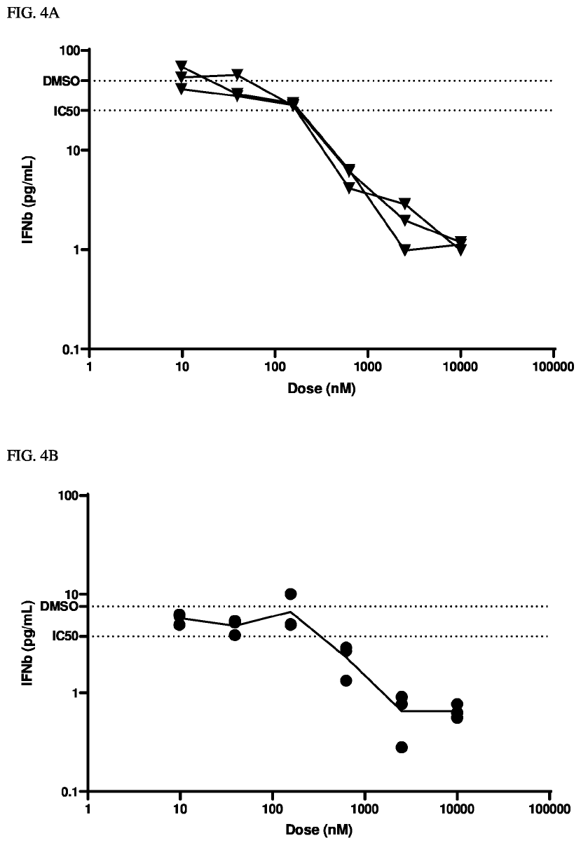

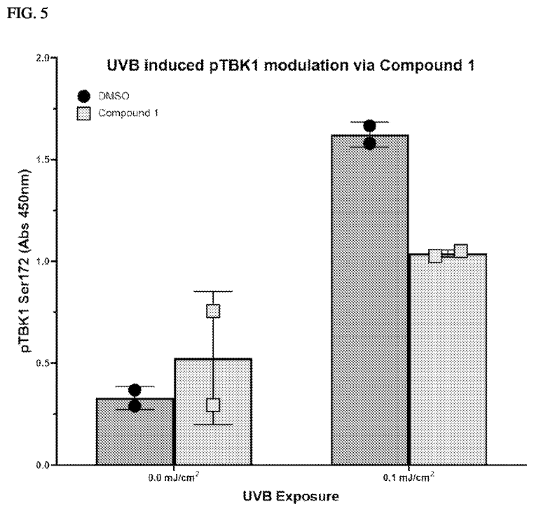

Stampidine was tested in this experiment. Results are shown in FIG. 4A and FIG. 4B. FIG. 4A represents a high responder (representative of PBMC's from 2 of the 6 donors tested) and FIG. 4B represents a moderate responder (representative of PBMC's from 3 of the 6 donors tested). One PBMC donor showed little to no effect from the excitation of the interferon response with decitabine (data not shown). Stampidine showed inhibition of IFN-β without any cellular toxicity. This provides support for the use of stampidine in the treatment of autoimmune disorders.

### Example 7—Analysis of UVB-Induced pTBK1 in HaCaT Cells

HaCaT cells were plated in 6-well plates at a density of ˜100 k/well in HaCaT media (DMEM, optimized 1× (Addex Bio)+1% pen strep (Gibco)+5% heat inactivated fetal bovine serum (Gibco)). The cells were then cultured at 37° C. overnight. The next day, the cells were treated with the test compounds. Each test compound was diluted and added to media aliquots to provide desired concentrations. To add the test compound+media mixture, an equivalent amount of media from each well was aspirated and then replenished with the media dosed with the test compound. The cells were then cultured for an additional 96 hours with compound treatment prior to UVB exposure.

The media was then aspirated from the wells, with the remaining cells at least 80% confluent in each well. One mL of PBS was then added to each well, and the plate was then placed under a UVB lamp. A UVB sensor was positioned near the plate to register the plate's exposure. The cells were exposed to the UVB light until they reached 0.1 mJ/cm². Then the plate was covered and transferred to a sterile hood for processing.

The PBS was aspirated out of the wells, and the wells were replenished with 3 mL fresh culture media. The cells were then cultured for an additional 24 hours, and samples were processed 24 hours post-UVB exposure. To process the samples, the media was aspirated, the plate placed on ice, and the cells washed with cold PBS, which was then aspirated off. Another 1 ml, of cold PBS was added to each well. The cells were then scraped in the cold PBS solution and transferred to conical tubes on ice. The cells were then spun at ≥1000 RCF at 4° C. for 5 minutes. The cells were then resuspended in 1 mL of cold PBS and transferred to a microcentrifuge tube. The cells were spun at ≥1000 RCF at 4° C. for another 5 minutes, and the PBS was aspirated off. The cell pellet was prepared for lysis.

A RIPA lysis buffer (#BP-115, Boston Bio-Products) was added to a Halt protease and phosphate inhibitor cocktail (#78440, ThermoFisher), and the mixture was cooled on ice. About 30 μL of the lysis buffer mix was added to the cells. The samples were briefly vortexed and then incubated on ice for at least 15 minutes. The cells were then spun ≥1000 RCF at 4° C. for 5 minutes and the supernantant was transferred to a clean tube. The protein concentration of the cell lysate was measured using Pierce™ Rapid Gold BCA Protein Assay Kit #AF3225 (ThermoFisher). ELISA analysis was run on select samples using one of the following kits:
a. FastScan™ Phospho-TBK1/NAK (Ser172) ELISA Kit #46948 (Cell Signaling Technologies)
b. FastScan™ Total TBK1/NAK ELISA Kit #15816 (Cell Signaling Technologies)
c. FastScan™ Phospho-STING (Ser366) ELISA Kit #82083 (Cell Signaling Technologies)
d. FastScan™ Phospho-IRF-3 (Ser396) ELISA Kit #50386 (Cell Signaling Technologies)
e. FastScan™ Total IRF-3 ELISA Kit #29771 (Cell Signaling Technologies)
f. Phospho-NAK/TBK1 (S172) ELISA Kit (ab279952) (Abcam)
g. Phospho-IRF3 (S386) ELISA Kit (ab279833) (Abcam)

### Results

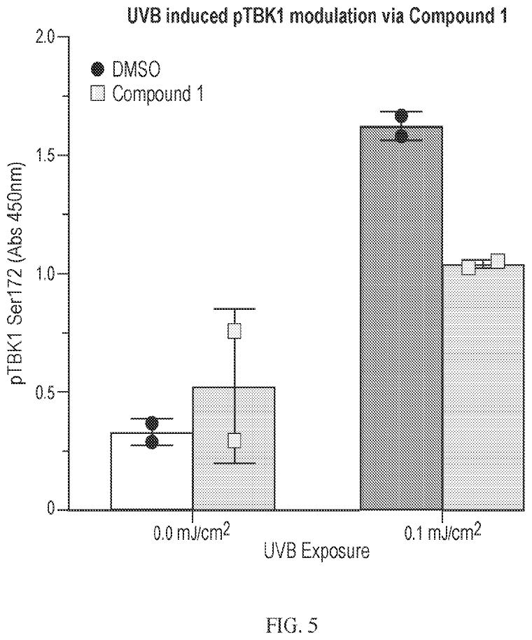

FIG. 5 shows pTBK1 levels in HaCaT cells treated with stampidine (labeled “Compound 1” in FIG. 5) or a DMSO control with, or without, UVB exposure. Stampidine showed a reduction of UVB-induced phosphorylated TANK-binding kinase 1 (pTBK1) after 96 hours of treatment. pTBK1 plays an essential role in regulating inflammatory responses. Reduction of pTBK1 levels after contact with stampidine provides evidence of a reduction in inflammation upon administration of stampidine, supporting its potential utility in treating autoimmune diseases as described herein.

### Example 8—Cellular Assay for Altering IFN Production in THP1 TREX1 KO Cells

THP1 Dual TREX1 KO cells were purchased from Invivogen. The THP1-Dual™ KO-TREX1 cells were cultured in RPMI 1640, 10% heat-inactivated fetal bovine serum, 25 mM HEPES, 10 μg/mL Blasticidin, and 100 μg/mL Zeocin. THP1-Dual™ KO-TREX1 cells were treated with a dose titration of test compound in the presence of 1 μM 5-aza-2′-deoxycytidine (Sigma, cat #189825). Type 1 Interferon and cell viability were assessed after five days of treatment.

Stock solution of test compound was prepared in DMSO followed by a three-fold dilution in DMSO. Additional 50× dilution was prepared in cell culture media for each dilution. 10 μL of diluted test compound was then added to a 384-well plate.

THP1-Dual™ KO-TREX1 cells were treated with 1 μM 5-aza-2′-deoxycytidine. THP1-Dual™ KO-TREX1 cells (50 μL) were added to each well of the 384-well plate containing test compound titration at 10,000 cells/well. Cells were incubated at 37° C., 5% CO₂ in a humidified incubator for five days. On day five, 20 μL of cell supernatant was transferred to a 384-well, white-walled plate, followed by addition to each well of 50 μL of QUANTI-LUC solution containing stabilizer. Luminescence was detected on a plate reader according to manufacturer's instructions. Percent inhibition of interferon was calculated using the following analysis: (Average DMSO-Sample)/(Average DMSO-Average 30 μM inhibition control reagent)*100.

The remaining cells were assessed for cell viability by adding 30 μl of CellTiter-Glo (Promega, 09683) solution to each well, and placed on a shaker for 10 minutes at room temperature. Luminescence was detected on a plate reader, according to manufacturer's instructions. Percent inhibition of cell viability using CellTiter-Glo was calculated using the following analysis: (Average DMSO-Sample)/(Average DMSO-Average 20 μM viability control reagent)*100. The viability control reagent was Z-Leu-Leu-leucinal (see, for example, https://pubchem.nchi.nlm.nih.gov/compound/462382).

### Part II—Results

Experimental results are provided in FIGS. 6A and 6B for stampidine (FIG. 6A) and stavudine (FIG. 6B) for changes in IFN levels as % inhibition versus compound concentration. CellTiter-Glo analysis demonstrated ˜15% reduction in cell viability at the highest stampidine dose and ˜10% reduction in cell viability at the highest stavudine dose.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating a disorder selected from the group consisting of an autoimmune disorder and a neurological disorder comprising administering to a subject in need thereof a therapeutically effective amount of a compound of Formula I to treat the disorder;
wherein Formula I is represented by: 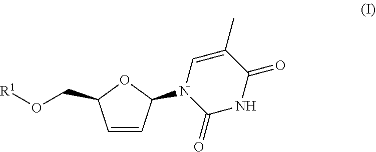
or a pharmaceutically acceptable salt thereof; wherein:
R¹ is 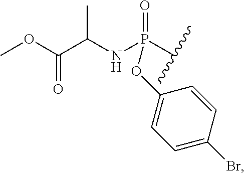 [SMILES: *P(=O)(NC(C)C(=O)OC)Oc1ccc(Br)cc1]
hydrogen, —P(O)(OH)₂, —P(O)(OH)—O—P(O)(OH)₂, or —(P(O)(OH))₂—O—P(O)(OH)₂.

2. The method of claim 1, wherein the compound of Formula I is administered in a pharmaceutical composition comprising the compound and a pharmaceutically acceptable carrier, adjuvant, and/or vehicle.

3. The method of claim 1 or 2, wherein the method further comprises administering an effective amount of an additional therapeutic agent.

4. A method of inhibiting LINE1 reverse transcriptase activity in a subject suffering from a disorder selected from the group consisting of an autoimmune disorder and a neurological disorder, comprising contacting a LINE1 reverse transcriptase with an effective amount of a compound of Formula I, in order to inhibit the activity of said LINE1 reverse transcriptase; wherein Formula I is represented by: 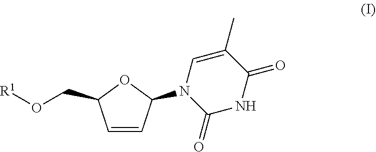
or a pharmaceutically acceptable salt thereof; wherein:
R¹ is 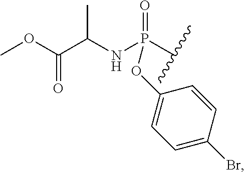 [SMILES: *P(=O)(NC(C)C(=O)OC)Oc1ccc(Br)cc1]
hydrogen, —P(O)(OH)₂, —P(O)(OH)—O—P(O)(OH)₂, or —(P(O)(OH))₂—O—P(O)(OH)₂.

5. The method of claim 4, wherein the method further comprises inhibiting HERV-K reverse transcriptase activity in the subject.

6. A method of inhibiting HERV-K reverse transcriptase activity in a subject suffering from a disorder selected from the group consisting of an autoimmune disorder and a neurological disorder, comprising contacting a HERV-K reverse transcriptase with an effective amount of a compound of Formula I, in order to inhibit the activity of said HERV-K reverse transcriptase; wherein Formula I is represented by: 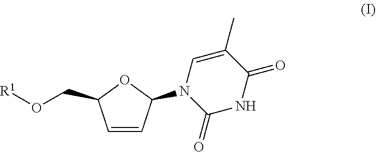
or a pharmaceutically acceptable salt thereof; wherein:
R¹ is 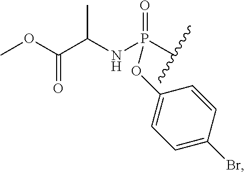 [SMILES: *P(=O)(NC(C)C(=O)OC)Oc1ccc(Br)cc1]
hydrogen, —P(O)(OH)₂, —P(O)(OH)—O—P(O)(OH)₂, or —(P(O)(OH))₂—O—P(O)(OH)₂.

7. The method of any one of claims 1-6, wherein the compound is a compound of Formula I.

8. The method of any one of claims 1-7, wherein R¹ is

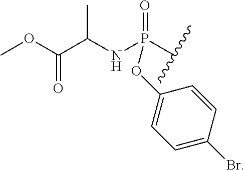 [SMILES: *P(=O)(NC(C)C(=O)OC)Oc1ccc(Br)cc1]

9. The method of any one of claims 1-7, wherein R¹ is hydrogen.

10. The method of any one of claims 1-7, wherein R¹ is —P(O)(OH)₂, —P(O)(OH)—O—P(O)(OH)₂, or —(P(O)(OH))₂—O—P(O)(OH)₂.

11. The method of any one of claims 1-6, wherein the compound is 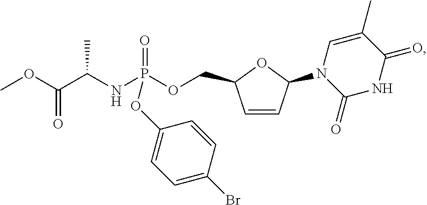 [SMILES: COC(=O)[C@H](C)NP(=O)(OC[C@@H]1C=C[C@H](n2cc(C)c(=O)[nH]c2=O)O1)Oc1ccc(Br)cc1]
or a pharmaceutically acceptable salt thereof.

12. The method of any one of claims 1-6, wherein the compound is 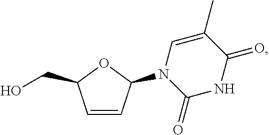 [SMILES: Cc1cn([C@H]2C=C[C@@H](CO)O2)c(=O)[nH]c1=O]
or a pharmaceutically acceptable salt thereof.

13. The method of any one of claims 1-12, wherein the disorder is an autoimmune disorder.

14. The method of claim 13, wherein the autoimmune disorder is selected from Aicardi-Goutieres syndrome, rheumatoid arthritis, psoriasis, systemic lupus erythematosus (SLE), cutaneous lupus erythematosus (CLE), graft versus host disease, scleroderma, type I diabetes, dermatomyositis, inflammatory bowel disease, ulcerative colitis, Crohn's disease, vasculitis, and Sjögren's syndrome.

15. The method of any one of claims 1-12, wherein the disorder is a neurological disorder.

16. The method of claim 15, wherein the neurological disorder is Alzheimer's disease, amyotrophic lateral sclerosis (ALS), multiple sclerosis, Parkinson's disease, Huntington's disease, peripheral neuropathy, age-related macular degeneration, Creutzfeldt-Jacob disease, stroke, prion disease, frontotemporal dementia, Pick's disease, progressive supranuclear palsy, spinocerebellar ataxias, Lewy body disease, dementia, multiple system atrophy, epilepsy, bipolar disorder, schizophrenia, an anxiety disorder, or major depression.

17. The method of any one of claims 1-16, wherein the subject has (i) elevated expression of LINE1 RNA, LINE1 ORF1 polypeptide, and/or LINE1 ORF2 polypeptide; and/or (ii) elevated activity of LINE1 reverse transcriptase.

18. The method of any one of claims 1-17, wherein the subject has (i) expression of HERV-K RNA and/or (ii) activity of HERV-K reverse transcriptase.

19. The method of any one of claims 1-18, wherein the subject is a human.
